(19) United States
(12) Patent Application Publication
(10) Pub. No.: US 2024/0277751 A1
(43) Pub. Date: Aug. 22, 2024

(54) CRYSTALLINE POLYMORPHIC FORMS OF STING AGONISTS ASSOCIATED WITH METAL IONS CAPABLE OF MODULATING AN IMMUNE RESPONSE

(71) Applicant: The Regents of the University of Michigan, Ann Arbor, MI (US)

(72) Inventors: James J. Moon, Ann Arbor, MI (US); Xiaoqi Sun, Ann Arbor, MI (US); Kyung Soo Park, Ann Arbor, MI (US); Xuehui Huang, Ann Arbor, MI (US)

(21) Appl. No.: 18/031,832

(22) PCT Filed: Oct. 15, 2021
(86) PCT No.: PCT/US2021/055212

§ 371 Date: Apr. 13, 2023

Related U.S. Application Data
(60) Provisional application No. 63/092,295, filed on Oct. 15, 2020.

(51) Int. Cl. A61K 31/7084; A61K 39/12; A61K 45/06; A61K 47/60; A61K 47/64

(52) U.S. Cl. CPC A61K 31/7084; A61K 39/12; A61K 45/06; A61K 47/60; A61K 47/6455; A61K 2039/572; C07B 2200/13

(57) ABSTRACT

This disclosure provides compositions and methods for stimulating the innate immune response in a subject with agents capable of stimulating an innate immune response in a subject upon administration to the subject. In particular, the present invention is directed to crystalline polymorphic forms (e.g. coordinate polymeric forms) of STING agonists associated (e.g. mixed) with one or more metal ions (e.g., Zn²⁺, Mn²⁺, Al³⁺, Fe³⁺, Cu²⁺), as well as systems and methods utilizing such compositions (e.g., in therapeutic settings).

### CROSS REFERENCE TO RELATED APPLICATIONS

This application claims benefit of priority to U.S. Provisional Application No. 63/092,295, filed Oct. 15, 2020, the contents of which are incorporated herein by reference in their entirety.

### STATEMENT REGARDING FEDERALLY SPONSORED RESEARCH OR DEVELOPMENT

This invention was made with government support under AI127070 awarded by the National Institutes of Health. The government has certain rights in the invention.

### FIELD OF THE INVENTION

This disclosure provides compositions and methods for stimulating the innate immune response in a subject with agents capable of stimulating an innate immune response in a subject upon administration to the subject. In particular, the present invention is directed to crystalline polymorphic forms (e.g., coordinate polymeric forms) of STING agonists associated (e.g., mixed) with one or more metal ions (e.g., Zn²⁺, Mn²⁺, Al³⁺, Fe³⁺, Cu²⁺), as well as systems and methods utilizing such compositions (e.g., in therapeutic settings).

### BACKGROUND OF THE INVENTION

Immune checkpoint blockades can allow patients' own immune system to fight against cancer. However, the current average response rate to immune check point blockades is only around 30%. This may be attributed to that some tumors, characterized as “cold tumors”, are less visible to the immune system. The characters of such tumors include low inflammatory responses, less mutation burden, and deficient tumoral-infiltration of T cells and other pro-inflammatory immune cells. In contrast, “hot tumors”, with more inflammatory signatures available for immune system recognize, have better therapeutic response rate to cancer immunotherapy. Therefore, it is critical to understand how to turn “cold tumors” into “hot tumors”.

Accumulating evidence indicates that immune surveillance of tumors, mediated by the innate immune system, recognizes the presence of tumor by sensing tumor cell-derived DNA by stimulator of interferon genes (STING) pathway. The activation of STING pathway could elicit innate immune cascade, such as type-I interferon response and other pro-inflammation phenotypic change, which further elicit adaptive antitumor reaction. Therefore, STING is regarded as the “trigger” of the reversion from “cold tumor” to “hot tumor”. For example, intra-tumoral administration of STING agonists could elicit antitumor immune response to both local and metastatic tumors. In a clinical setting, type-1 interferon response is found to be a signature of better cancer therapy prognosis similar to antigen-specific T cells infiltration. Therefore, developing STING agonists with great in-vivo stability, favorable pharmacokinetics properties and acceptable safety profiles is of great significance and high translational value.

The vast majority of STING agonists under clinical development are cyclic dinucleotides (CDN) and their derivatives. Importantly, conventional nucleotide-based STING agonists have several major limitations. For example, STING agonists in clinical trials now are administrated via intratumoral injection due to their poor pharmaceutical properties, including low bioavailability and off-target toxicity. For example, the half-life of MIW815 in serum is only ˜16 min, and over 98% of MIW815 entered the systemic circulation 1 hour after intratumor injection in a phase I clinical trial. This precludes their applicability for the treatment of metastatic cancer. In addition, nucleotide-based STING agonists generally do not cross the cell membrane, thus limiting activation of intracellular STING.

Therefore, there is an urgent need to find a simple and translational formulation for delivery of CDN STING agonists.

Developing new polymorphs has been Widely used to improve the performance of drugs, which may provide benefits such as better efficacy, solubility, shelf life, or ease of manufacture, and eventually translate into increased adoption of the drug. New polymorphs of CDN STING agonists have not been reported yet. Discovery of such new polymorphs or polymeric forms of CDN STING agonists is very promising to improve their pharmaceutical properties and solve its formulation and delivery problems.

The present invention addresses these needs.

### SUMMARY

Experiments conducted during the course of developing embodiments for the present invention resulted in the surprising discovery that specific metal ions (Zn²⁺, Mn²⁺, Al³⁺, Fe³⁺, Cu²⁺) could coordinate with CDN based STING agonists and form new coordination structures or crystalline polymorphic forms (e.g., coordinated polymeric forms). In particular, such experiments demonstrated that CDN STING agonists mixed with Zn²⁺ and/or Mn²⁺ crystalline polymorphic forms (e.g., coordinated polymeric forms) capable of assembling into homogeneous and stable nanoparticles in the presence of poly-histidine₃₃ (H33)-polyethylene glycol (PEG). Compositions comprising crystalline polymorphic forms (e.g., coordinated polymeric forms) of STING agonists mixed with Zn²⁺ and/or Mn²⁺ are indicated for use to improve the pharmaceutical properties and therapeutic effect of the CDN STING agonist. Such compositions are generally formed by self-assembly in water by simple admixing, thus expediting the manufacturing process and clinical translation. Such results have significant clinical importance, as these compositions comprising crystalline polymorphic forms (e.g., coordinated polymeric forms) of STING agonists mixed with Zn²⁺ and/or Mn²⁺ can induce immune responses against specific tumors through systemic administration thereby avoiding the need for direct local injection into tumors.

Accordingly, such results and embodiments indicate a new class of drug delivery systems for both local and systemic delivery of agents capable of stimulating an innate immune response in a subject upon administration to the subject.

As such, this disclosure provides compositions and methods for stimulating the innate immune response in a subject with agents capable of stimulating an innate immune response in a subject upon administration to the subject. In particular, the present invention is directed to crystalline polymorphic forms (e.g., coordinated polymeric forms) of STING agonists associated (e.g., complexed, conjugated, encapsulated, absorbed, adsorbed, admixed) with one or more metal ions (e.g., Zn²⁺, Mn²⁺, Al³⁺, Fe³⁺, Cu²⁺), as well as systems and methods utilizing such compositions (e.g., in diagnostic and/or therapeutic settings).

Accordingly, in certain embodiments, the present invention provides compositions comprising crystalline polymorphic forms (e.g., coordinated polymeric forms) of one or more STING agonists mixed with one or more metal ions (e.g., Zn²⁺, Mn²⁺, Al³⁺, Fe³⁺, Cu²⁺). In some embodiments, such compositions of one or more STING agonists mixed with one or more metal ions (e.g., Zn²⁺, Mn²⁺, Al³⁺, Fe³⁺, Cu²⁺) are further associated with poly(histidine)-PEG (eg., H33-PEG).

Such compositions are not limited to particular ratio between the one or more STING agonists and the one or more metal ions (e.g., Zn²⁺, Mn²⁺, Al³⁺, Fe³⁺, Cu²⁺). In some embodiments, the amount of the one or more STING agonist within the crystalline polymorphic forms (e.g., coordinated polymeric forms) is between 0.01 and 5 mg/ml. In some embodiments, the molar ratio of one or more metal ions (e.g., Zn²⁺, Mn²⁺, Al³⁺, Fe³⁺, Cu²⁺) to the one or more STING agonists should be more than 0.1. In some embodiments wherein the crystalline polymorphic forms (e.g., coordinated polymeric forms) are associated with poly(histidine)-PEG (eg., H33-PEG), the concentration of H33-PEG should be more than 1.3 mg/ml for effective formulation. In some embodiments, the length of PEG is between 1 to 500 repeat units. In some embodiments, the length of polyhistidine is between 1 to 50 repeat units.

Such compositions are not limited to a particular type or kind of STING agonist. In some embodiments, the STING agonist is a small molecular agonist of STING. In some embodiments, the small molecular agonists of STING are cyclic dinucleotides. For example, in some embodiments, the cyclic dinucleotides include cGAMP, cdiAMP, cdiGMP, and cAIMP. Additional examples of cyclic purine dinucleotides are described in some detail in, e.g., U.S. Pat. Nos. 7,709,458 and 7,592,326; WO2007/054279; and Yan et al., Bioorg. Med. Chem Lett. 18: 5631 (2008), each of which is hereby incorporated by reference. In some embodiments, additional STING agonists are selected from 5,6-Dimethylxanthenone-4-acetic acid (DMXAA), methoxyvone, 6,4′-dimethoxyflavone, 4′-methoxyflavone, 3′,6′-dihydroxyflavone, 7,2′-dihydroxyflavone, daidzein, formononetin, and retusin 7-methyl ether, or any derivatives thereof. In some embodiments, the small molecular agonists of STING include, but are not limited to, 2′3′-cGAMP, 3′3′-cGAMP, c-di-AMP, c-di-GMP, cAIMP, cAIMP Difluor, cAIM(PS)2, Difluor (Rp/Sp), 2′2′cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, SB11285, STING-agonist-C11, STING agonist-1, STING agonist G10, and Gemcitabine.

In some embodiments, the small molecular agonist of STING is selected from

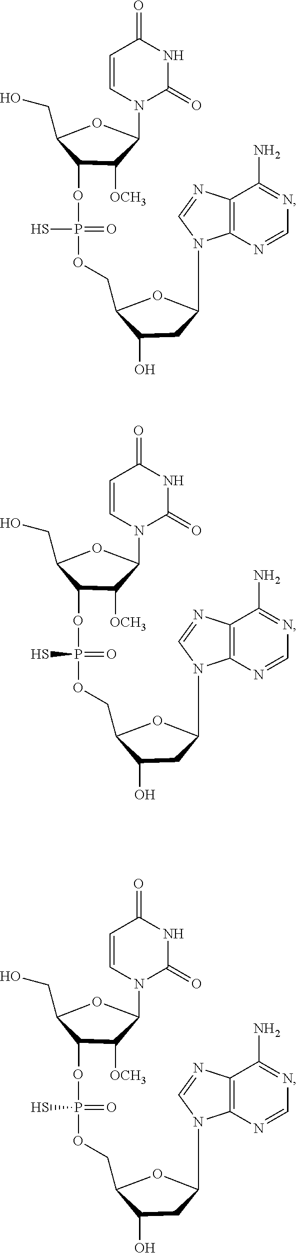

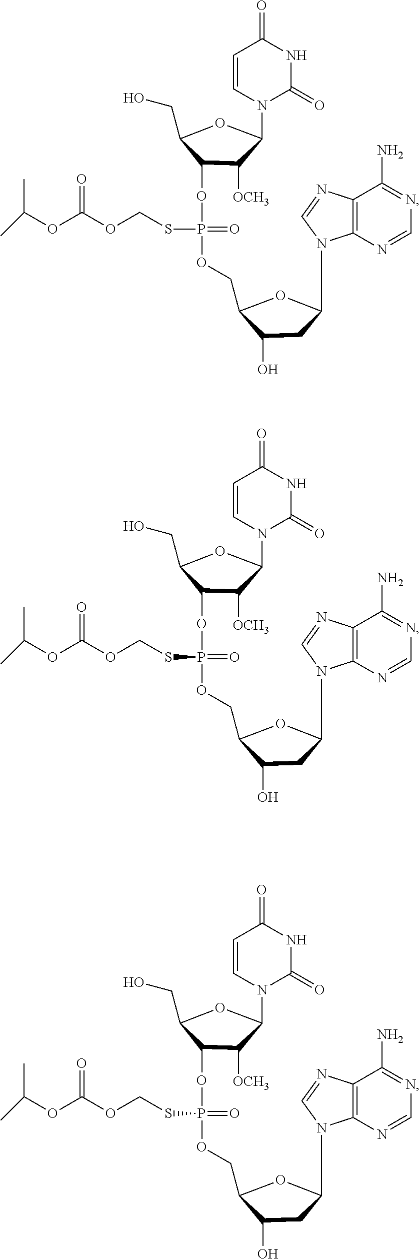

### SB11285 (Spring Bank Pharmaceuticals), Gemcitabine

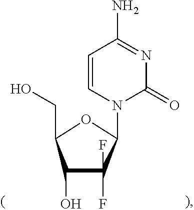

### STING-agonist-C11

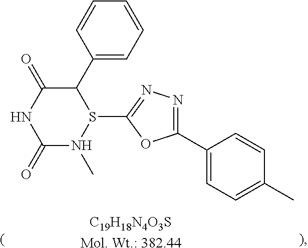

STING agonist-1

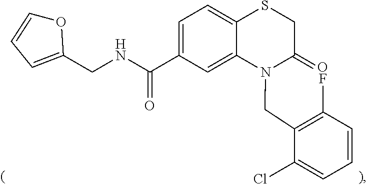

STING agonist G10

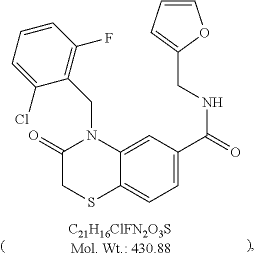

2′3′-cGAMP, 3′3′-cGAMP c-di-AMP, c-di-GMP, cAIMP, cAIMP Difluor, cAIM(PS)2, Difluor (Rp/Sp), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, cGAMP, 2′3′-cGAMP, 2′2′-cGAMP, 3′3′-cGAMP, cGAM(PS)2, 2′3′-cGAM(PS)2(Rp/Sp), 2′2′-cGAM(PS)2, 2′3′-cGAM(PS)2, cGAMP Fluorinated, 3′3′-cGAMP Fluorinated, 2′3′-cGAMP Fluorinated, 2′2′-cGAMP Fluorinated, c-di-AMP, 2′3′-cdAMP, 2′2′-cdAMP, 3′3′-cdAMP, c-di-AM(PS)2, 2′3′-c-di-AM(PS)2 (Rp,Rp), 2′2′-c-di-AM(PS)2, 3′3′-c-di-AM(PS)2, c-di-AMP Fluorinated, 2′3′-cdAMP Fluorinated, 2′2′-cdAMP Fluorinated, 3′3′-cdAMP Fluorinated, cdGMP, 2′3′-cdGMP, 2′2′-cdGMP, 3′3′-cdGMP, c-di-GM(PS)2, 2′3′-c-di-GM(PS)2, 2′2′-c-di-GM(PS)2, 3′3′-c-di-GM(PS)2, cdGMP Fluorinated, 2′3′-cdGMP Fluorinated, 2′2′-cdGMP Fluorinated, 3′3′-cdGMP Fluorinated, cAIMP, 2′3′-cAIMP, 2′2′-cAIMP, 3′3′-cAIMP, cAIMP Difluor (3′3′-cAIMP Fluorinated, 2′3′-cAIMP Fluorinated, 2′2′-cAIP Fluorinated, cAIM (PS)2 Difluor, 3′3′-cAIM(PS)2 Difluor (Rp/Sp), 2′3′-cAIM(PS)2 Difluor, 2′2′-cAIM(PS)2 Difluor, c-di-IMP, 2′3′-cdIMP, 2′2′-cdIMP, 3′3′-cdIMP, c-di-IM(PS)2, 2′3′-c-di-IM(PS)2, 2′2′-c-di-IM(PS)2, 3′3′-c-di-TM(PS)2, c-di-IMP Fluorinated, 2′3′-cdIMP Fluorinated, 2′2′-cdIMP Fluorinated, and 3′3′-cdIMP Fluorinated, amidobenzimidazole (ABZI)-based compounds, SR-717-based compounds, and MSA-2-based compounds.

In some embodiments, the STING agonist mixed with one or more metal ions (e.g., Zn²⁺, Mn²⁺, Al³⁺, Fe³⁺, Cu²⁺) is further modified into a prodrug form. For example, in some embodiments, a prodrug of a STING agonist is a STING agonist attached with hydrophobic moieties that assist with loading into nanoparticles and/or assist with tissue retention. In some embodiments, the STING agonists are modified with a cleavable lipid moiety to render the crystalline polymorphic forms (e.g., coordinated polymeric forms) of the STING agonist mixed one or more metal ions (e.g., Zn²⁺, Mn²⁺, Al³⁺, Fe³⁺, Cu²⁺) into a prodrug.

In some embodiments, the agent is a STING activating compound (see, e.g., WO2017011920, WO2017027646, WO2017011622, U.S. Patent Application Publication No. 20160287623, WO2016100261, U.S. Patent Application Publication No. 20160074507, and WO2015161762).

In some embodiments, STING agonists combined with pharmaceutically active components are provided (see, e.g., STING activation/chemotherapy (WO2016096577), STING activation/selected vaccine formulation stimulating an immune response (U.S. Patent Application Publication Nos. 20150056224 and 20140205653), and STING activation/cytokines production (WO2013185052)).

As noted, in some embodiments, the crystalline polymorphic forms (e.g., coordinated polymeric forms) of one or more STING agonists mixed with one or more metal ions (e.g., Zn²⁺, Mn²⁺, Al³⁺, Fe³⁺, Cu²⁺) are further associated with poly(histidine)-PEG (eg., H33-PEG).

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), and monomethoxypolyethylene glycol-imidazolyl-carbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present invention. The disclosures of these patents are herein incorporated by reference in their entirety for all purposes. In addition, monomethoxypolyethyleneglycolacetic acid (MePEG-CH₂COOH) is particularly useful for preparing PEG-lipid conjugates including, e.g., PEG-DAA conjugates.

The PEG moiety of the PEG-H33 conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In preferred embodiments, the PEG moiety has an average molecular weight of about 2,000 daltons or about 750 daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In a preferred embodiment, the linker moiety is a non-ester containing linker moiety. As used herein, the term “non-ester containing linker moiety” refers to a linker moiety that does not contain a carboxylic ester bond (—OC(O)—). Suitable non-ester containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, as well as combinations thereof (such as a linker containing both a carbamate linker moiety and an amido linker moiety). In a preferred embodiment, a carbamate linker is used to couple the PEG to the lipid.

In other embodiments, an ester containing linker moiety is used to couple the PEG to H33. Suitable ester containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form a PEG/H33 conjugate. Such phosphatidylethanolamines are commercially available, or can be isolated or synthesized using conventional techniques known to those of skilled in the art. Phosphatidylethanolamines containing saturated or unsaturated fatty acids with carbon chain lengths in the range of C₁₀ to C₂₀ are preferred. Phosphatidylethanolamines with mono- or diunsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoylphosphatidylethanolamine (DOPE), and distearoyl-phosphatidylethanolamine (DSPE).

In some embodiments, the compositions comprising one or more STING agonists mixed with one or more metal ions (e.g., Zn²⁺, Mn²⁺, Al³⁺, Fe³⁺, Cu²⁺) are further associated (e.g., complexed, conjugated, encapsulated, absorbed, adsorbed, admixed) with an adjuvant.

In some embodiments, the adjuvant is selected from the group consisting of CPG, polyIC, poly-ICLC, 1018 ISS, aluminum salts (for example, aluminum hydroxide, aluminum phosphate), Amplivax, BCG, CP-870,893, CpG7909, CyaA, dSLIM, Cytokines (such as GM-CSF, IL-2, IFN-a, Flt-3L), IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, imiquimod, resiquimod, gardiquimod, 3M-052, SRL172, Virosomes and other Virus-like particles, YF-17D, VEGF trap, beta-glucan, Pam3Cys, Aquila's QS21 stimulon, vadimezan, AsA404 (DMXAA), 3M MED19197, glucopyranosyl lipid adjuvant (GLA), GLA-SE, CDld ligands (such as C20:2, OCH, AH04-2, α-galatosylceramide, α-C-galatosylceramide, α-mannosylceramide, α-fructosylceramide, β-galatosylceramide, β-mannosylceramide), STING agonists (e.g. cyclic dinucleotides, including Cyclic [G(3′,5′)pA(3′,5′)p], Cyclic [G(2′,5′)pA(3′,5′)p], Cyclic [G(2′,5′)pA(2′,5′)p], Cyclic diadenylate monophosphate, Cyclic diguanylate monophosphate), CL401, CL413, CL429, Flagellin, RC529, E6020, imidazoquinoline-based small molecule TLR-7/8a (including its lipidated analogues), virosomes, AS01, AS02, AS03, AS04, AS15, IC31, CAF01, ISCOM, Cytokines (such as GM-CSF, IL-2, IFN-α, Flt-3L), and bacterial toxins (such as CT, and LT). In some embodiments, the adjuvant is any derivative of an adjuvant (e.g., cholesterol-modified CpG) or any combinations thereof. In some embodiments, the adjuvant is a dendritic cell targeting molecule.

In some embodiments, the compositions comprising one or more STING agonists mixed with one or more metal ions (e.g., Zn²⁺, Mn²⁺, Al³⁺, Fe³⁺, Cu²⁺) are further associated (e.g., complexed, conjugated, encapsulated, absorbed, adsorbed, admixed) with one or more neo-antigenic peptides, wherein each of the one or more neo-antigenic peptides is specific for a neo-antigenic mutation identified from a neoplasia biological sample obtained from a subject. In some embodiments, the subject is a human being.

In some embodiments, the one or more neo-antigenic peptides range from about 5 to about 50 amino acids in length. In some embodiments, the one or more neo-antigenic peptides range from about 15 to about 35 amino acids in length. In some embodiments, the one or more neo-antigenic peptides range from about 18 to about 30 amino acids in length. In some embodiments, the one or more neo-antigenic peptides range from about 6 to about 15 amino acids in length.

In some embodiments, the compositions comprising one or more STING agonists mixed with one or more metal ions (e.g., Zn²⁺, Mn²⁺, Al³⁺, Fe³⁺, Cu²⁺) are further associated (e.g., complexed, conjugated, encapsulated, absorbed, adsorbed, admixed) with one or more biomacromolecule agents. Such compositions are not limited to a particular biomacromolecule agent.

In some embodiments, the biomacromolecule agent is a nucleic acid. Such embodiments encompass any type of nucleic acid molecule including, but not limited to, RNA, siRNA, microRNA, interference RNA, mRNA, replicon mRNA, RNA-analogues, and DNA.

In some embodiments, the biomacromolecule agent is a peptide.

In some embodiments, the peptide is Adrenocorticotropic Hormone (ACTH), a growth hormone peptide, a Melanocyte Stimulating Hormone (MSH), Oxytocin, Vasopressin, Corticotropin Releasing Factor (CRF), a CRF-related peptide, a Gonadotropin Releasing Hormone Associated Peptide (GAP), Growth Hormone Releasing Factor (GRF), Lutenizing Hormone Release Hormone (LH-RH), an orexin, a Prolactin Releasing Peptide (PRP), a somatostatin, Thyrotropin Releasing Hormone (THR), a THR analog, Calcitonin (CT), a CT-precursor peptide, a Calcitonin Gene Related Peptide (CGRP), a Parathyroid Hormone (PTH), a Parathyroid Hormone Related Protein (PTHrP), Amylin, Glucagon, Insulin, an Insulin-like peptide, NeuroPeptide Y (NPY), a Pancreatic Polypeptide (PP), Peptide YY (PYY), Cholecystokinin (CCK), a CCK-related peptide, Gastrin Releasing Peptide (GRP), Gastrin, a Gastrin-related peptide, a Gastrin inhibitory peptide, Motilin, Secretin, Vasoactive Intestinal Peptide (VIP), a VIP-related peptide, an Atrial-Natriuretic Peptide (ANP), a Brain Natriuretic Peptide (BNP), a C-Type Natriuretic Peptide(CNP), a tachykinin, an angiotensin, a renin substrate, a renin inhibitor, an endothelin, an endothelin-related peptide, an opioid peptide, a thymic peptide, an adrenomedullin peptide, an allostatin peptide, an amyloid beta-protein fragment, an antimicrobial peptide, an antioxidant peptide, an apoptosis related peptide, a Bag Cell Peptide (BCPs), Bombesin, a bone Gla protein peptide, a Cocaine and Amphetamine Related Transcript (CART) peptide, a cell adhesion peptide, a chemotactic peptide, a complement inhibitor, a cortistatin peptide, a fibronectin fragment, a fibrin related peptide, FMRF, a FMRF amide-related peptide (FaRP), Galanin, a Galanin-related peptide, a growth factor, a growth factor-related peptide, a G-Therapeutic Peptide-Binding Protein fragment, Gualylin, Uroguanylin, an Inhibin peptide, Interleukin (IL), an Interleukin Receptor protein, a laminin fragment, a leptin fragment peptide, a leucokinin, Pituitary Adenylate Cyclase Activating Polypeptide (PAPCAP), Pancreastatin, a polypeptide repetitive chain, a signal transducing reagent, a thrombin inhibitor, a toxin, a trypsin inhibitor, a virus-related peptide, an adjuvant peptide analog, Alpha Mating Factor, Antiarrhythmic Peptide, Anorexigenic Peptide, Alpha-1 Antitrypsin, Bovine Pineal Antireproductive Peptide, Bursin, C3 Peptide P16, Cadherin Peptide, Chromogranin A Fragment, Contraceptive Tetrapeptide, Conantokin G, Conantokin T, Crustacean Cardioactive Peptide, C-Telopeptide, Cytochrome b588 Peptide, Decorsin, Delicious Peptide, Delta-Sleep-Inducing Peptide, Diazempam-Binding Inhibitor Fragment, Nitric Oxide Synthase Blocking Peptide, OVA Peptide, Platelet Calpain Inhibitor (P1), Plasminogen Activator Inhibitor 1, Rigin, Schizophrenia Related Peptide, Sodium Potassium Atherapeutic Peptidase Inhibitor-1, Speract, Sperm Activating Peptide, Systemin, a Thrombin receptor agonist, Tuftsin, Adipokinetic Hormone, Uremic Pentapeptide, Antifreeze Polypeptide, Tumor Necrosis Factor (TNF), Leech [Des Asp10]Decorsin, L-Ornithyltaurine Hydrochloride, P-Aminophenylacetyl Tuftsin, Ac-Glu-Glu-Val-Val-Ala-Cys-pNA (SEQ ID NO: 1), Ac-Ser-Asp-Lys-Pro (SEQ ID NO: 2), Ac-rfwink-NH2, Cys-Gly-Tyr-Gly-Pro-Lys-Lys-Lys-Arg-Lys-Val-Gly-Gly (SEQ ID NO: 3), D-Ala-Leu, D-D-D-D-D (SEQ ID NO: 4), D-D-D-D-D-D (SEQ ID NO: 5), N-P-N-A-N-P-N-A (SEQ ID NO: 6), V-A-I-T-V-L-V-K (SEQ ID NO: 7), V-G-V-R-V-R (SEQ ID NO: 8), V-I-H-S(SEQ ID NO: 9), V-P-D-P-R (SEQ ID NO: 10), Val-Thr-Cys-Gly (SEQ ID NO: 11), R—S—R, Sea Urchin Sperm Activating Peptide, a SHU-9119 antagonist, a MC3-R antagonist, a MC4-R antagonist, Glaspimod, HP-228, Alpha 2-Plasmin Inhibitor, APC Tumor Suppressor, Early Pregnancy Factor, Gamma Interferon, Glandular Kallikrei N-1, Placental Ribonuclease Inhibitor, Sarcolecin Binding Protein, Surfactant Protein D, Wilms' Tumor Suppressor, GABAB 1b Receptor Peptide, Prion Related Peptide (iPRP13), Choline Binding Protein Fragment, Telomerase Inhibitor, Cardiostatin Peptide, Endostatin Derived Peptide, Prion Inhibiting Peptide, N-Methyl D-Aspartate Receptor Antagonist, and C-PeptideAnalog.

In some embodiments, the peptide is selected from 177Lu-DOTA0-Tyr3-Octreotate, Abarelix acetate, ADH-1, Afamelanotidec, melanotan-1, CUV1647, Albiglutide, Aprotinin, Argipressin, Atosiban acetate, Bacitracin, Bentiromide, a BH3 domain, Bivalirudin, Bivalirudin trifluoroacetate hydrate, Blisibimod, Bortezomib, Buserelin, Buserelin acetate, Calcitonin, Carbetocin, Carbetocin acetate, Cecropin A and B, Ceruletide, Ceruletide diethylamine, Cetrorelix, Cetrorelix acetate, Ciclosporine, Cilengitidec, EMD121974, Corticorelin acetate injection, hCRF, Corticorelin ovine triflutate, corticorelin trifluoroacetate, Corticotropin, Cosyntropin, ACTH 1-24, tetracosactide hexaacetate, Dalbavancin, Daptomycin, Degarelix acetate, Depreotide trifluoroacetate (plus sodium pertechnetate), Desmopressin acetate, Desmopressin DDAVP, Dulaglutide, Ecallantide, Edotreotide (plus yttrium-90), Elcatonin acetate, Enalapril maleate (or 2-butanedioate), Enfuvirtide, Eptifibatide, Exenatide, Ganirelix acetate, Glatiramer acetate, Glutathion, Gonadorelin, Gonadorelin acetate, GnRH, LHRH, Goserelin, Goserelin acetate, Gramicidin, Histrelin acetate, Human calcitonin, Icatibant, Icatibant acetate, IM862, oglufanide disodium, KLAKLAK, Lanreotide acetate, Lepirudin, Leuprolide, Leuprolide acetate, leuprorelin, Liraglutide, Lisinopril, Lixisenatide, Lypressin, Magainin2, MALP-2Sc, macrophage-activating lipopeptide-2 synthetic, Nafarelin acetate, Nesiritide, NGR-hTNF, Octreotide acetate, Oritavancin, Oxytocin, Pasireotide, Peginesatide, Pentagastrin, Pentetreotide (plus indium-111), Phenypressin, Pleurocidin, Pramlintide, Protirelin, thyroliberin, TRH, TRF, Salmon calcitonin, Saralasin acetate, Secretin (human), Secretin (porcine), Semaglutide, Seractide acetate, ACTH, corticotropin, Sermorelin acetate, GRF 1-29, Sinapultide, KL4 in lucinactant, Sincalide, Somatorelin acetate, GHRH, GHRF, GRF, Somatostatin acetate, Spaglumat magnesium (or sodium) salt, Substance P, Taltirelin hydrate, Teduglutide, Teicoplanin, Telavancin, Teriparatide, Terlipressin acetate, Tetracosactide, Thymalfasin, thymosin a-1, Thymopentin, Trebananib, Triptorelin, Triptorelin pamoate, Tyroserleutide, Ularitide, Vancomycin, Vapreotide acetate, Vasoactive intestinal peptide acetate, Vx-001c, TERT572Y, Ziconotide acetate, α5-α6 Bax peptide, and β-defensin.

In some embodiments, the peptide is any peptide which would assist in achieving a desired purpose with the composition. For example, in some embodiments, the peptide is any peptide that will facilitate treatment of any type of disease and/or disorder.

In some embodiments, the peptide is an antigen. In some embodiments, the antigen is selected from the group consisting of a peptide based antigen, a protein based antigen, a polysaccharide based antigen, a saccharide based antigen, a lipid based antigen, a glycolipid based antigen, a nucleic acid based antigen, an inactivated organism based antigen, an attenuated organism based antigen, a viral antigen, a bacterial antigen, a parasite antigen, an antigen derived from an allergen, and a tumor antigen. In some embodiments, the antigen is any type of viral, bacterial or self-antigen including, but not limited to, FimH against urinary tract infection; soluble F protein from respiratory syncytial virus (RSV); NEF, GAG, and ENV protein from HIV; Streptococcus pneumoniae proteins; HMGB1 protein; hemagglutinin and neuroamidase protein against influenza; Viral antigens derived from HPV type 16 and 18; gL2, ICP4, gD2ΔTMR, gD2ΔTMR, or ICP4.2 from HSV-2; antigens from S. pneumoniae, such as a pneumolysoid, Choline-binding protein A (CbpA), or Pneumococcal surface protein A (PspA), SP1912, SP1912, SP1912L, SP0148 with or without a signal sequence, SP2108 with or without a signal sequence; Antigens from Chlamydia trachomatis, such as a CT209 polypeptide antigen, a CT253 polypeptide antigen, a CT425 polypeptide antigen, a CT497 polypeptide antigen, and a CT843 polypeptide antigen; amyloid-beta peptide. In some embodiments, the antigen is conjugated to the outer surface of the composition. In some embodiments, the antigen is encapsulated within the composition.

Such compositions are not limited to a particular manner of generating crystalline polymorphic forms (e.g., coordinated polymeric forms) of a STING agonist mixed with one or more metal ions (e.g., Zn²⁺, Mn²⁺, Al³⁺, Fe³⁺, Cu²⁺). In some embodiments, association between a STING agonist and one or more metal ions (e.g., Zn²⁺, Mn²⁺, Al³⁺, Fe³⁺, Cu²⁺) resulting in crystalline polymorphic forms (e.g., coordinated polymeric forms) of a STING agonist mixed with one or more metal ions (e.g., Zn²⁺, Mn²⁺, Al³⁺, Fe³⁺, Cu²⁺) is accomplished during a “one-pot” reaction. The term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, a one-pot reaction occurs wherein a STING agonist is reacted with one or more metal ions (e.g., Zn²⁺, Mn²⁺, Al³⁺, Fe³⁺, Cu²⁺) in one vessel, such conjugation being facilitated by the additional presence of H33-PEG.

In certain embodiments, the compositions comprising crystalline polymorphic forms (e.g., coordinated polymeric forms) of one or more STING agonists mixed with one or more metal ions (e.g., Zn²⁺, Mn²⁺, Al³⁺, Fe³⁺, Cu²⁺) are capable of stimulating an innate immune response in a subject upon administration to a subject. As such, in certain embodiments, the present invention provides methods for stimulating an innate immune response in a subject comprising administration of therapeutically effective amount of such a composition (e.g., crystalline polymorphic forms (e.g., coordinated polymeric forms) of a STING agonist mixed with one or more metal ions (e.g., Zn²⁺, Mn²⁺, Al³⁺, Fe³⁺, Cu²⁺) to a subject. In some embodiments, the innate immune response is an innate cytokine response mediated through cytokines in the subject. In some embodiments, the innate cytokine response is mediated through type 1 interferon in the subject.

In some embodiments, additional therapeutic agents are co-administered with such compositions. Examples of such therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, parenteral gold or oral gold.

In some embodiments, the compositions comprising one or more STING agonists mixed with one or more metal ions (e.g., Zn²⁺, Mn²⁺, Al³⁺, Fe³⁺, Cu²⁺) are further administered with one or more of an adjuvant (as described herein), a chemotherapeutic agent, an anti-immunosuppressive agent, an immunostimulatory agent, and an antigen (as described herein).

In some embodiments, the immunostimulatory agent is selected from anti-CTLA-4 antibody, anti-PD-1, anti-PD-L1, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, and inhibitors of IDO.

In some embodiments, the chemotherapeutic agent is selected from aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (TAXOL), pilocarpine, prochloroperazine, rituximab, tamoxifen, taxol, topotecan hydrochloride, trastuzumab, vinblastine, vincristine and vinorelbine tartrate.

In certain embodiments, the present invention provides methods of treating, preventing and/or ameliorating the symptoms of cancer in a subject (e.g., a human subject) through administration of a therapeutically effective amount of such a composition (e.g., crystalline polymorphic forms (e.g., coordinated polymeric forms) of a STING agonist mixed with one or more metal ions (e.g., Zn²⁺, Mn²⁺, Al³⁺, Fe³⁺, Cu²⁺) to the subject. In some embodiments, the subject is suffering from or at risk of suffering from cancer. In some embodiments, the compositions are used to elicit an immune response for vaccine applications. In some embodiments, the compositions are capable of stimulating an innate immune response in at least one cancer cell upon administration to the subject, wherein the subject is suffering from cancer. In some embodiments, stimulating an innate immune response comprises stimulating an innate cytokine response mediated through cytokines. In some embodiments, the innate cytokine response is mediated through type 1 interferon.

STING agonists have two key limitations for use as a cancer therapeutic: 1) poor pharmacokinetics and serious off-target side effects. Regarding poor pharmacokinetics, if administrated via intratumor injection, such STING agonists would easily diffuse away because of the small molecule weight and high hydrophilicity; if administrated via intravenous injection, such STING agonists would show low bioavailability to tumor tissue due to in-vivo instability, low lipophilicity and fast excretion. Regarding serious off-target side effects, as an immunological sensor to virus infections, STING is widely distributed across body. As such, high dose of STING agonists or systemically administrated STING agonists would nonspecifically activate the innate immune system and cause cytokine storm. The present invention addresses such limitations through providing crystalline polymorphic forms (e.g., coordinated polymeric forms) of such STING agonists mixed with one or more metal ions (e.g., Zn²⁺, Mn²⁺, Al³⁺, Fe³⁺, Cu²⁺).

In some embodiments, the compositions comprising one or more STING agonists mixed with one or more metal ions (e.g., Zn²⁺, Mn²⁺, Al³⁺, Fe³⁺, Cu²⁺) are further associated with (e.g., complexed, conjugated, encapsulated, absorbed, adsorbed, admixed) with one or more agents configured to target cancer cells. In some embodiments, the agent configured to target cancer cells is a tumor antigen selected from the group consisting of alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pm1-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGS), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), human EGFR protein or its fragments, such as human EGFR residues 306-325 (SCVRACGADSYEMEEDGVRK (SEQ ID NO: 12)) and residues 897-915 (VWSYGVTVWELMTFGSKPY (SEQ ID NO: 13)), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, TPS, WT1 (and WT1-derived peptide sequences: WT1 126-134 (RMFP NAPYL (SEQ ID NO: 14)), WT1 122-140 (SGQARMFPNAPYLPSCLES (SEQ ID NO: 15)), and WT1 122-144 (SGQARMFPNAPYLPSCLESQPTI (SEQ ID NO: 16)), MUC1 (and MUC1-derived peptides and glycopeptides such as RPAPGS (SEQ ID NO: 17), PPAHGVT (SEQ ID NO: 18), and PDTRP (SEQ ID NO: 19)), LMP2, EGFRvIII, Idiotype, GD2, Ras mutant, p53 mutant, Proteinase3 (PR1), Survivin, hTERT, Sarcoma translocation breakpoints, EphA2, EphA4, LMW-PTP, PAP, ML-IAP, AFP, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, sLe(animal), CYP1B1, PLAC1, GM3, BORIS, Tn, GloboH, NY-BR-1, RGS5, SART3, STn, Carbonic anhydrase IX, PAX5, OY-TES1, Sperm protein 17, LCK, HMWMAA, AKAP-4, XAGE 1, B7H3, Legumain, Tie 2, Page4, VEGFR2, MAD-CT-1, FAP, PDGFR-alpha, PDGFR-β, MAD-CT-2, Fos-related antigen 1, ERBB2, Folate receptor 1 (FOLR1 or FBP), IDH1, IDO, LY6K, fins-related tyro-sine kinase 1 (FLT1, best known as VEGFR1), KDR, PADRE, TA-CIN (recombinant HPV16 L2E7E6), SOX2, aldehyde dehydrogenase, and any derivative thereof. In some embodiments, the one or more agents configured to target cancer cells are conjugated to the outer surface of the composition. In some embodiments, the one or more agents configured to target cancer cells are encapsulated within the composition.

Such methods are not limited to a particular manner of administration. In some embodiments, the administration is systemic administration. In some embodiments, the administration is local administration.

In some embodiments, the composition is co-administered with a chemotherapeutic agent. In some embodiments, the chemotherapeutic agent is one or more of the following: aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (TAXOL), pilocarpine, prochloroperazine, rituximab, tamoxifen, taxol, topotecan hydrochloride, trastuzumab, vinblastine, vincristine and vinorelbine tartrate.

In some embodiments, the cancer is one or more selected from bladder cancer, brain cancer, breast cancer, cervical cancer, ovarian cancer, colo-rectal cancer, esophageal cancer, kidney cancer, liver cancer, lung cancer, nasopharangeal cancer, pancreatic cancer, prostate cancer, skin cancer, stomach cancer, gastric cancer, head and neck cancer, testicular cancer, melanoma, acute myelogenous leukemia, chronic myelogenous leukemia, chronic lymphocytic leukemia, T cell lymphocytic leukemia, and B cell lymphomas, and uterine cancer.

In certain embodiments, the present invention provides methods of treating, preventing and/or ameliorating the symptoms of a viral infection in a subject (e.g., a human subject) through administration of a therapeutically effective amount of such a composition (e.g., crystalline polymorphic forms (e.g., coordinated polymeric forms) of a STING agonist mixed with one or more metal ions (e.g., Zn²⁺, Mn²⁺, Al³⁺, Fe³⁺, Cu²⁺) to the subject. In some embodiments, the subject is suffering from or at risk of suffering from conditions and symptoms caused by a viral infection (e.g., COVID-19). In some embodiments, the compositions comprising crystalline polymorphic forms (e.g., coordinated polymeric forms) of one or more STING agonists mixed with one or more metal ions (e.g., Zn²⁺, Mn²⁺, Al³⁺, Fe³⁺, Cu²⁺) are used to reduce levels of ACE2 and SARS-COV-2 virus cell entry. In some embodiments, the compositions are used to inhibit SARS-COV2 S protein induced NF-kB activation. In some embodiments, the compositions are used to reduce proinflammatory cytokine release by immune effector cells. In some embodiments, the compositions are used to inhibit endothelial activation and dysfunction. Such methods are not limited to a particular manner of administration. In some embodiments, the administration is systemic administration. In some embodiments, the administration is local administration.

In some embodiments, the viral infection is a SARS-CoV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virals having M^{pro} protease activity and/or expression.

In some embodiments, the one or more symptoms related to viral infection includes, but is not limited to, fever, fatigue, dry cough, myalgias, dyspnea, acute respiratory distress syndrome, and pneumonia.

Such methods are not limited to a particular manner of administration. In some embodiments, the administration is systemic administration. In some embodiments, the administration is local administration.

In some embodiments, the present invention provides methods for treating, ameliorating and/or preventing acute respiratory distress syndrome and/or pneumonia in a subject, comprising administering to the subject such a composition (e.g., crystalline polymorphic forms (e.g., coordinated polymeric forms) of a STING agonist mixed with one or more metal ions (e.g., Zn²⁺, Mn²⁺, Al³⁺, Fe³⁺, Cu²⁺). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments for preventing, attenuating or treating a subject having or at risk for having conditions and symptoms caused by a viral infection (e.g., COVID-19), the composition is co-administered with one or more of the following therapeutic agents: remdesivir, dexamethasone, and hydroxychloroquine. Such methods are not limited to a particular manner of administration. In some embodiments, the administration is systemic administration. In some embodiments, the administration is local administration.

### BRIEF DESCRIPTION OF THE DRAWINGS

FIG. 1. Unique coordination polymeric forms were found after mixing CDA with certain metal ions, including Mn²⁺, Al³⁺, Fe³⁺, Cu²⁺, and Zn²⁺. Scale bar=200 μm.

FIG. 2. Large crystals, composed of CDA and Zn²⁺ were formed when Zn²⁺/CDA was greater than 0.1. Unspecified scale bar=100 μm.

FIG. 3. Large crystals, composed of CDA, Zn²⁺ and Mn²⁺, were formed when (Zn²⁺+Mn²⁺)/CDA was greater than 0.1. Unspecified scale bar=100 μm.

FIG. 4. Nanoscale coordination structures were synthesized by mixing CDA, Zn²⁺, Mn²⁺ and H33-PEG20K with stirring. The resulting nanostructures were finely tuned by changing the poly-histidine length, PEG length, and metal ions.

FIG. 5. Orders of addition of CDA, Zn²⁺, Mn²⁺, and H33-PEG20K while stirring determine the yield of the resulting formulation.

FIG. 6. Orthogonal experiment 1 to optimize the formulation efficacy. Formulation were not efficiently generated by adding H33-PEG to CDA/M²⁺ mixture, followed by washing with PBS.

FIG. 7. Orthogonal experiment 2 to optimize the formulation efficiency. Formulations were efficiently generated by adding M²⁺ to CDA/H33-PEG20K mixture in specific ratios, followed by washing with PBS.

FIG. 8. Dynamic light scattering, drug release, and in vivo therapeutic effects of CDA-Zn/Mn@H33-PEG20K formulation.

FIG. 9. CDA-Mn/Zn@H33-PEG admixed with S1 protein from SARS-CoV-2 generated robust S1-specific IgG, IgG1, and IgG2a antibody responses in BABL/c mice. Animals were immunized on days 0, 14, and 28. Antibody titers were measured on days 14 (“1”), 28 (“2”), 42 (“3”), and 70 (“4”).

FIG. 10. CDA-Mn/Zn@H33-PEG admixed with RBD protein from SARS-CoV-2 generated robust RBD-specific IFN-gamma+ T-cell responses in BABL/c mice. Animals were immunized on days 0, 14, and 28. ELISPOT assay was performed on day 35.

FIG. 11. One-pot formulation of STING agonist in CZMP. a) CDA, H33-PEG20k, and Zn2+/Mn²⁺ were mixed in order under continuous stirring in a one-pot synthesis. b) TEM image of the resulted CZMP nanoparticle. c) DLS size and zeta potential of CZMP. d) Release kinetics of CDA, Mn and Zn from CZMP in PBS media.

FIG. 12. One-pot STING agonist NP induces effectively deliver STING agonist to immune cells for STING activation. a) CZMP increased dendritic cell activation. b) CZMP increased the production of type-I IFNs and other pro-inflammatory cytokines. c) CZMP increased cellular uptake of STING agonist. d) Confocal imaging of subcellular distribution of CZMP shows effective endosome escape.

FIG. 13. One-pot STING agonist NP exerts robust anti-tumor efficacy after IT injection in CT 26 model. a) BALB/c mice were inoculated at S.C. flank with 3×10⁵ CT26 tumor cells. 5 pg CDA in free form or CZMP were injected I.T. on days 11, 14, 17, and 21. b) Serum cytokines were measured by ELISA at 6 h post the second dose. c, e) Tumor growth (c, e) and animal survival (e) were monitored over time. d, f) NK cell frequency in blood (d) and NK cell activation in tumor draining lymph node (f) were analyzed by flow cytometry. Data represent mean±SEM, from a representative experiment from 2 independent experiments with n=5 (b), n=5-7 (c-f). *P<0.05, **P<0.01, ***P<0.001, ****P<0.0001, analyzed by (d,f) one-way ANOVA or (e) two-way ANOVA with Bonferroni's multiple comparisons test, or (e) log-rank (Mantel-Cox) test.

FIG. 14. One-pot STING agonist NP exerts robust anti-tumor efficacy after IV injection. a-f) Therapeutic effects of CZMP on CT26 tumors after I.V. administration. a) BALB/c mice were inoculated at S.C. flank with 3×10⁵ CT26 tumor cells. 20 μg CDA in free form or CZMP were injected I.V. on days 11, 14, 17, and 21. b) Serum cytokines were measured by ELISA at 6 h post the second dose. c, e) Tumor growth (c, e) and animal survival (e) were monitored over time. d, f) NK cell frequency in blood (d) and NK cell activation in tumor draining lymph node (f) were analyzed by flow cytometry. g-i) Therapeutic effects of CMPCDA administered I.V. on B16F10 tumors. C57BL/6 mice were inoculated at S.C. flank with 3×105 B16F10 tumor cells. 20 μg CDA in free form or CZMP were injected I.V. on days 11, 14, 17, and 21 (g). h) Serum cytokines were measured by ELISA at 6 h post the second dose. i) tumor growth and survival were monitored over time. Data represent mean±SEM, from a representative experiment from 2 independent experiments with n=5 (b-), n=5-7 (c-f, h-i). *P<0.05, **P<0.01, ***P<0.001, ****P<0.0001, analyzed by (d,f) one-way ANOVA or (e,i) two-way ANOVA with Bonferroni's multiple comparisons test, or (e, i) log-rank (Mantel-Cox) test.

FIG. 15. One-pot STING agonist NP for COVID-19 vaccine delivery. Top panel: loading of Receptor-binding domain (RBD) of SARS-CoV2 in CZMP. Bottom panel: Lymph node draining of CZMP after S.C. administration.

FIG. 16. In-vivo immune response of CZMP-RBD for COVID-19 vaccination. a-b, f) balb/c mice were vaccinated three times at tail base with 2 weeks intervals. 2 weeks after each vaccination and 4 and 8 weeks after the last vaccination, sera samples were collected for antibody titer measurement by ELISA (b) and viral neutralization assay (f). c-d) C57BL mice were vaccinated three times at tail base with 2 weeks intervals. Spleens were harvested 1 week after the last vaccination for ELISPOT assay and germinal center formation analysis.

### DEFINITIONS

To facilitate an understanding of the present invention, a number of terms and phrases are defined below: As used herein, the term “complexed” as used herein relates to the non-covalent interaction of a biomacromolecule agent (e.g., antigen, adjuvant, etc) with a nanoparticle and/or microparticle.

As used herein, the term “conjugated” as used herein indicates a covalent bond association between a a biomacromolecule agent (e.g., antigen, adjuvant, etc) and a nanoparticle and/or microparticle.

As used herein, the term “encapsulated” refers to the location of a biomacromolecule agent (e.g., antigen, adjuvant, etc) that is enclosed or completely contained within the inside of a nanoparticle and/or microparticle.

As used herein, the term “absorbed” refers to a biomacromolecule agent (e.g., antigen, adjuvant, etc) that is taken into and stably retained in the interior, that is, internal to the outer surface, of a nanoparticle and/or microparticle.

As used herein, the term “adsorbed” refers to the attachment of a biomacromolecule agent (e.g., antigen, adjuvant, etc) to the external surface of a nanoparticle and/or microparticle. Such adsorption preferably occurs by electrostatic attraction. Electrostatic attraction is the attraction or bonding generated between two or more oppositely charged or ionic chemical groups. Generally, the adsorption is typically reversible.

As used herein, the term “admixed” refers to a biomacromolecule agent (e.g., antigen, adjuvant, etc) that is dissolved, dispersed, or suspended in a nanoparticle and/or microparticle. In some cases, the biomacromolecule agent may be uniformly admixed in the nanoparticle and/or microparticle.

As used herein, the terms “biological biomacromolecule” or “biomacromolecule” or “biomacromolecule agent” as used herein refer to a molecule with a molecular mass exceeding 1 kDa which can be isolated from an organism or from cellular culture, e.g., eukaryotic (e.g., mammalian) cell culture or prokaryotic (e.g., bacterial) cell culture. In some embodiments, the use of the term refers to polymers, e.g., biopolymers such as nucleic acids (including, but not limited to, RNA, siRNA, microRNA, interference RNA, mRNA, replicon mRNA, RNA-analogues, DNA, etc.), polypeptides (such as proteins), carbohydrates, and lipids. In some embodiments, the term “biomacromolecule” refers to a protein. In some embodiments, the term “biomacromolecule” refers to a recombinant protein or a fusion protein. In some embodiments, the protein is soluble. In some embodiments, the biomacromolecule is an antibody, e.g., a monoclonal antibody. In some embodiments, the biomacromolecule is an adjuvant, an antigen, a therapeutic agent, an imaging agent, etc.

As used herein, the term “antigen” is defined herein as a molecule which contains one or more epitopes that will stimulate a hosts immune system to make a cellular antigen-specific immune response, and/or a humoral antibody response. Antigens can be peptides, proteins, polysaccharides, saccharides, lipids, nucleic acids, and combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components. An antigen may be an oligonucleotide or polynucleotide which expresses an antigen. Antigens can be natural or synthetic antigens, for example, haptens, polyepitopes, flanking epitopes, and other recombinant or synthetically derived antigens (see, e.g., Bergmann, et al., Eur. J. Immunol., 23:2777-2781 (1993); Bergmann, et al., J. Immunol., 157:3242-3249 (1996); Suhrbier, Immunol. and Cell Biol., 75:402-408 (1997)).

As used herein, the term “neo-antigen” or “neo-antigenic” means a class of tumor antigens that arises from a tumor-specific mutation(s) which alters the amino acid sequence of genome encoded proteins.

As used herein, the term “tumor-specific antigen” is defined herein as an antigen that is unique to tumor cells and does not occur in or on other cells in the body.

As used herein, the term “tumor-associated antigen” is defined herein as an antigen that is not unique to a tumor cell and is also expressed in or on a normal cell under conditions that fail to induce an immune response to the antigen.

As used herein, the term “adjuvant” is defined herein as a substance increasing the immune response to other antigens when administered with other antigens. Adjuvants are also referred to herein as “immune potentiators” and “immune modulators”.

As used herein, the term “antigen-presenting cells” are defined herein as highly specialized cells that can process antigens and display their peptide fragments on the cell surface together with molecules required for lymphocyte activation. The major antigen-presenting cells for T cells are dendritic cells, macrophages and B cells. The major antigen-presenting cells for B cells are follicular dendritic cells.

As used herein, the term “cross-presentation” is defined herein as the ability of antigen-presenting cells to take up, process and present extracellular antigens with MHC class I molecules to CD8 T cells (cytotoxic T cells). This process induces cellular immunity against most tumors and against viruses that do not infect antigen-presenting cells. Cross-presentation is also required for induction of cytotoxic immunity by vaccination with protein antigens, for example in tumor vaccination.

As used herein, the terms “immunologic”, “immunological” or “immune” response is the development of a humoral and/or a cellular response directed against an antigen.

As used herein, the term “kit” refers to any delivery system for delivering materials. In the context of the compostions as described herein (e.g., compositions comprising crystalline polymorphic forms (e.g., coordinated polymeric forms) of STING agonists associated (e.g., complexed, conjugated, encapsulated, absorbed, adsorbed, admixed) with one or more metal ions (e.g., Zn²⁺, Mn²⁺, Al³⁺, Fe³⁺, Cu²⁺)), such delivery systems include systems that allow for the storage, transport, or delivery of such compositions and/or supporting materials (e.g., written instructions for using the materials, etc.) from one location to another. For example, kits include one or more enclosures (e.g., boxes) containing the neccary agents and/or supporting materials. As used herein, the term “fragmented kit” refers to delivery systems comprising two or more separate containers that each contain a subportion of the total kit components. The containers may be delivered to the intended recipient together or separately. For example, a first container may contain a composition comprising a STING agonist, while a second container contains one or more metal ions (e.g., Zn²⁺, Mn²⁺, Al³⁺, Fe³⁺, Cu²⁺). Indeed, any delivery system comprising two or more separate containers that each contains a subportion of the total kit components are included in the term “fragmented kit.” In contrast, a “combined kit” refers to a delivery system containing all of the components necessary to synthesize and utilize any of the crystalline polymorphic forms (e.g., coordinated polymeric forms) of STING agonists associated (e.g., complexed, conjugated, encapsulated, absorbed, adsorbed, admixed) with one or more metal ions (e.g., Zn²⁺, Mn²⁺, Al³⁺, Fe³⁺, Cu²⁺) as described (e.g., in a single box housing each of the desired components). The term “kit” includes both fragmented and combined kits.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “drug” or “therapeutic agent” is meant to include any molecule, molecular complex or substance administered to an organism for diagnostic or therapeutic purposes, including medical imaging, monitoring, contraceptive, cosmetic, nutraceutical, pharmaceutical and prophylactic applications. The term “drug” is further meant to include any such molecule, molecular complex or substance that is chemically modified and/or operatively attached to a biologic or biocompatible structure.

As used herein, the term “solvent” refers to a medium in which a reaction is conducted. Solvents may be liquid but are not limited to liquid form. Solvent categories include but are not limited to nonpolar, polar, protic, and aprotic.

### DETAILED DESCRIPTION OF THE INVENTION

Experiments conducted during the course of developing embodiments for the present invention resulted in the surprising discovery that specific metal ions (e.g., Zn²⁺, Mn²⁺, Al³⁺, Fe³⁺, Cu²⁺) could coordinate with CDN based STING agonists and form new crystalline polymorphic forms (e.g., coordinated polymeric forms). In particular, such experiments demonstrated that CDN STING agonists mixed with Zn²⁺ and/or Mn²⁺ form crystalline polymorphic forms (e.g., coordinated polymeric forms) capable of assembling into homogeneous and stable nanoparticles in the presence of poly-histidine₃₃ (H33) and polyethylene glycol (PEG). Compositions comprising crystalline polymorphic forms (e.g., coordinated polymeric forms) of STING agonists mixed with one or more metal ions (e.g., Zn²⁺, Mn²⁺, Al³⁺, Fe³⁺, Cu²⁺) are indicated for use to improve the pharmaceutical properties and therapeutic effect of the CDN STING agonist. Such compositions are generally formed by self-assembly in water by simple admixing, thus expediting the manufacturing process and clinical translation. Such results have significant clinical importance, as these compositions comprising crystalline polymorphic forms (e.g., coordinated polymeric forms) of STING agonists mixed with one or more metal ions (e.g., Zn²⁺, Mn²⁺, Al³⁺, Fe³⁺, Cu²⁺) can induce immune responses against specific tumors through systemic administration thereby avoiding the need for direct local injection into tumors.

Accordingly, such results and embodiments indicate a new class of drug delivery systems for both local and systemic delivery of agents capable of stimulating an innate immune response in a subject upon administration to the subject.

As such, this disclosure provides compositions and methods for stimulating the innate immune response in a subject with agents capable of stimulating an innate immune response in a subject upon administration to the subject. In particular, the present invention is directed to crystalline polymorphic forms (e.g., coordinated polymeric forms) of STING agonists associated (e.g., complexed, conjugated, encapsulated, absorbed, adsorbed, admixed) with one or more metal ions (e.g., Zn²⁺, Mn²⁺, Al³⁺, Fe³⁺, Cu²⁺), as well as systems and methods utilizing such compositions (e.g., in diagnostic and/or therapeutic settings).

Accordingly, in certain embodiments, the present invention provides compositions comprising crystalline polymorphic forms (e.g., coordinated polymeric forms) of one or more STING agonists mixed with one or more metal ions (e.g., Zn²⁺, Mn²⁺, Al³⁺, Fe³⁺, Cu²⁺). In some embodiments, such crystalline polymorphic forms (e.g., coordinated polymeric forms) of one or more STING agonists mixed with one or more metal ions (e.g., Zn²⁺, Mn²⁺, Al³⁺, Fe³⁺, Cu²⁺) further associated with poly(histidine)-PEG (eg., H33-PEG).

Such compositions are not limited to particular ratio between the one or more STING agonists and the one or more metal ions (e.g., Zn²⁺, Mn²⁺, Al³⁺, Fe³⁺, Cu²⁺). In some embodiments, the amount of the one or more STING agonist within the crystalline polymorphic forms (e.g., coordinated polymeric forms) is between 0.01 and 5 mg/ml. In some embodiments, the molar ratio of one or more metal ions (e.g., Zn²⁺, Mn²⁺, Al³⁺, Fe³⁺, Cu²⁺) to the one or more STING agonists should be more than 0.1. In some embodiments wherein the crystalline polymorphic forms (e.g., coordinated polymeric forms) are associated with H33-PEG, the concentration of H33-PEG should be more than 1.3 mg/ml for effective formulation. In some embodiments, the length of PEG is between 1 to 500 repeat units. In some embodiments, the length of polyhistidine is between 1 to 50 repeat units.

Such compositions are not limited to a particular type or kind of STING agonist. In some embodiments, the STING agonist is a small molecular agonist of STING. In some embodiments, the small molecular agonists of STING are cyclic dinucleotides. For example, in some embodiments, the cyclic dinucleotides include cGAMP, cdiAMP, cdiGMP, and cAIMP. Additional examples of cyclic purine dinucleotides are described in some detail in, e.g., U.S. Pat. Nos. 7,709,458 and 7,592,326; WO2007/054279; and Yan et al., Bioorg. Med. Chem Lett. 18: 5631 (2008), each of which is hereby incorporated by reference. In some embodiments, additional STING agonists are selected from 5,6-Dimethylxanthenone-4-acetic acid (DMXAA), methoxyvone, 6,4′-dimethoxyflavone, 4′-methoxyflavone, 3′,6′-dihydroxyflavone, 7,2′-dihydroxyflavone, daidzein, formononetin, and retusin 7-methyl ether, or any derivatives thereof. In some embodiments, the small molecular agonists of STING include, but are not limited to, 2′3′-cGAMP, 3′3′-cGAMP, c-di-AMP, c-di-GMP, cAIMP, cAIMP Difluor, cAIM(PS)2, Difluor (Rp/Sp), 2′2′-cGAMP, 2′3′-cGAM(PS)2 (Rp/Sp), 3′3′-cGAMP Fluorinated, c-di-AMP Fluorinated, 23-c-di-AMP, 2′3-c-di-AM(PS)2 (Rp,Rp), c-di-GMP Fluorinated, 2′3′-c-di-GMP, c-di-IMP, SB11285, STING-agonist-C 11, STING agonist-1, STING agonist G10 and Gemcitabine.

In some embodiments, the small molecular agonist of STING is selected from

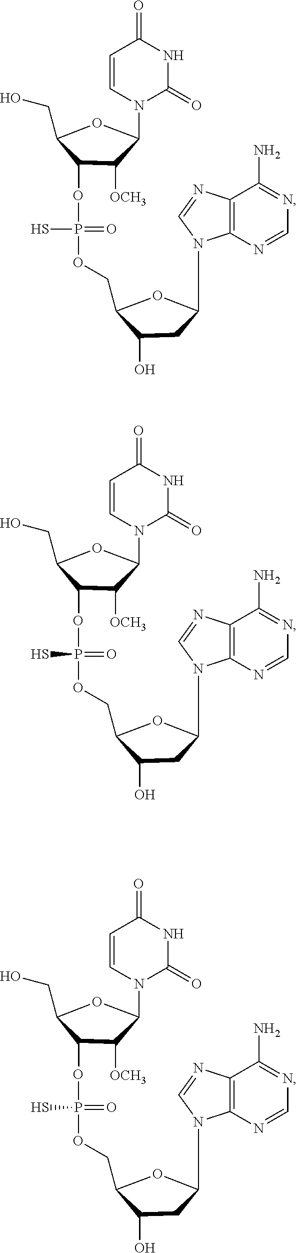

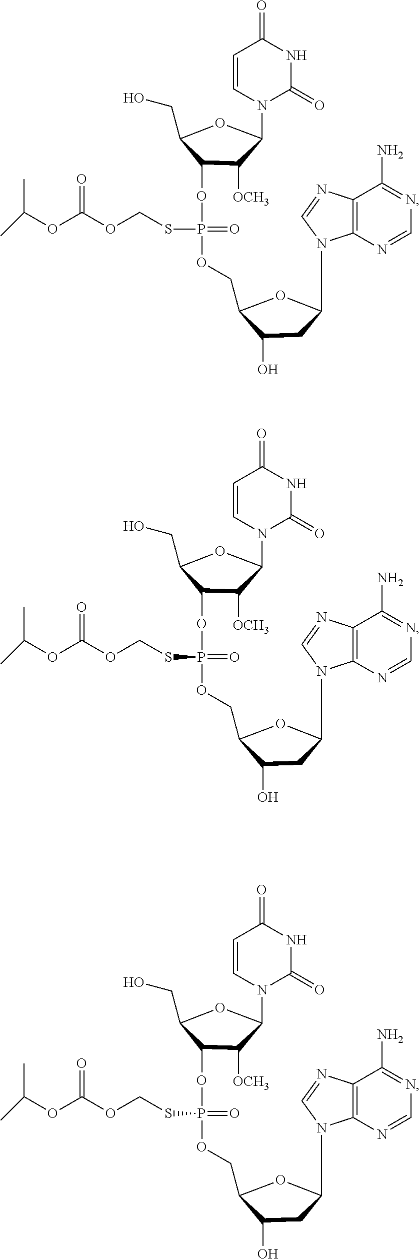

### SB11285 (Spring Bank Pharmaceuticals) Gemcitabine

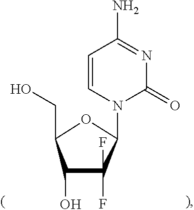

STING agonist-C11

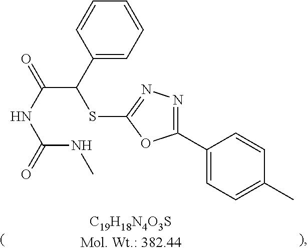 [SMILES: CNC(=O)NC(=O)C(Sc1nnc(-c2ccc(C)cc2)o1)c1ccccc1 ; "C19H18N4O3S" ; "Mol. Wt.: 382.44"]

STING agonist-1

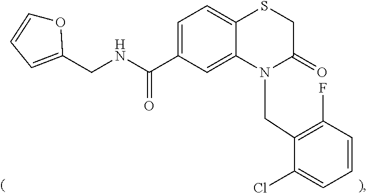 [SMILES: O=C(NCc1ccco1)c1ccc2c(c1)N(Cc1c(F)cccc1Cl)C(=O)CS2]

STING agonist G10

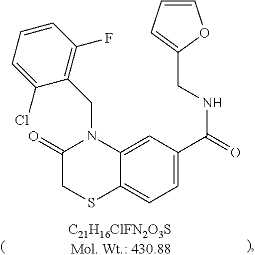

2′3′-cGAMP, 3′3′cGAMP, c-di-AMP, c-di-GMP, cAIMP, cAIMP Difluor, cAIM(PS)2, Difluor (Rp/Sp), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, cGAMP, 2′3′-cGAMP, 2′2′-cGAMP, 3′3′-cGAMP, cGAM(PS)2, 2′3′-cGAM(PS)2(Rp/Sp), 2′2′-cGAM(PS)2, 2′3′-cGAM(PS)2, cGAMP Fluorinated, 3′3′-cGAMP Fluorinated, 2′3′-cGAMP Fluorinated, 2′2′-cGAMP Fluorinated, c-di-AMP, 2′3′-cdAMP, 2′2′-cdAMP, 3′3′-cdAMP, c-di-AM(PS)2, 2′3′-c-di-AM(PS)2 (Rp,Rp), 2′2′-c-di-AM(PS)2, 3′3′-c-di-AM(PS)2., c-di-AMP Fluorinated, 2′3′-cdAMP Fluorinated, 2′2′-cdAMP Fluorinated, 3′3′-cdAMP Fluorinated, cdGMP, 2′3′-cdGMP, 2′2%-cdGMP, 3′3′-cdGMP, c-di-GM(PS)2, 2′3′-c-di-GM (PS)2, 2′2′-c-di-GM(PS)2, 3′3′-c-di-GM(PS)2, cdGMP Fluorinated, 2′3′-cdGMP Fluorinated, 2′2′-cdGMP Fluorinated, 3′3′-cdGMP Fluorinated, cAIMP, 2′3′-cAIMP, 2′2′-cAIMP, 3′3′-cAIMP, cAIMP Difluor (3′3′-cAIMP Fluorinated, 2′3′-cAIMP Fluorinated, 2′2′-cAIMP Fluorinated, cAIM(PS)2 Difluor, 3′3′-cAIM(PS)2 Difluor (Rp/Sp), 2′3′-cAIM(PS)2 Difluor, 2′2′-c AIM(PS)2 Difluor, c-di-IMP, 2′3′-cdIMP, 2′2′-cdIMP, 3′3′-cdIMP, c-di-IM (PS)2, 2′3′-c-di-IM(PS)2, 2′2′-c-di-IM(PS)2, 3′3′-c-di-IM(PS)2, c-di-IMP Fluorinated, 2′3′-cdIMP Fluorinated, 2′2′-cdIMP Fluorinated, and 3′3′-cdIMP Fluorinated, amidobenzimidazole (ABZI)-based compounds, SR-717-based compounds, and MSA-2-based compounds.

In some embodiments, the STING agonist mixed with one or more metal ions (e.g., Zn²⁺, Mn²⁺, Al³⁺, Fe³⁺, Cu²⁺) is further modified into a prodrug form. For example, in some embodiments, a prodrug of a STING agonist is a STING agonist attached with hydrophobic moieties that assist with loading into nanoparticles and/or assist with tissue retention. In some embodiments, the STING agonists are modified with a cleavable lipid moiety to render the crystalline polymorphic forms (e.g., coordinated polymeric forms) of the STING agonist mixed one or more metal ions (e.g., Zn²⁺, Mn²⁺, Al³⁺, Fe³⁺, Cu²⁺) into a prodrug.

In some embodiments, the agent is a STING activating compound (see, e.g., WO2017011920, WO2017027646, WO2017011622, U.S. Patent Application Publication No. 20160287623, WO2016100261, U.S. Patent Application Publication No. 20160074507, and WO2015161762).

In some embodiments, STING agonists combined with pharmaceutically active components are provided (see, e.g., STING activation/chemotherapy (WO2016096577), STING activation/selected vaccine formulation stimulating an immune response (U.S. Patent Application Publication Nos. 20150056224 and 20140205653), and STING activation/cytokines production (WO2013185052)).

As noted, in some embodiments, the crystalline polymorphic forms (e.g., coordinated polymeric forms) of one or more STING agonists mixed with one or more metal ions (e.g., Zn²⁺, Mn²⁺, Al³⁺, Fe³⁺, Cu²⁺) are further associated with poly(histidine)-PEG (eg., H33-PEG).

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), and monomethoxypolyethylene glycol-imidazolyl-carbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present invention. The disclosures of these patents are herein incorporated by reference in their entirety for all purposes. In addition, monomethoxypolyethyleneglycolacetic acid (MePEG-CH₂COOH) is particularly useful for preparing PEG-lipid conjugates including, e.g., PEG-DAA conjugates.

The PEG moiety of the PEG-H33 described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In preferred embodiments, the PEG moiety has an average molecular weight of about 2,000 daltons or about 750 daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In a preferred embodiment, the linker moiety is a non-ester containing linker moiety. As used herein, the term “non-ester containing linker moiety” refers to a linker moiety that does not contain a carboxylic ester bond (—OC(O)—). Suitable non-ester containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, as well as combinations thereof (such as a linker containing both a carbamate linker moiety and an amido linker moiety). In a preferred embodiment, a carbamate linker is used to couple the PEG to H33.

In other embodiments, an ester containing linker moiety is used to couple the PEG to H33. Suitable ester containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the H33 conjugate. Such phosphatidylethanolamines are commercially available, or can be isolated or synthesized using conventional techniques known to those of skilled in the art.

Phosphatidylethanolamines containing saturated or unsaturated fatty acids with carbon chain lengths in the range of C₁₀ to C₂₀ are preferred. Phosphatidylethanolamines with mono- or diunsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoylphosphatidylethanolamine (DOPE), and distearoyl-phosphatidylethanolamine (DSPE).

In some embodiments, the compositions are further associated with an adjuvant. Such embodiments are not limited to a particular type of adjuvant. Generally, adjuvants are any substance whose admixture into the composition increases or otherwise modifies the immune response to an antigen. Carriers are scaffold structures, for example a polypeptide or a polysaccharide, to which the antigenic peptide (e.g., neo-antigenic peptide) is capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response.

Suitable adjuvants include, but are not limited to 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel® vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Several immunological adjuvants (e.g., MF59) specific for dendritic cells and their preparation have been described previously (Dupuis M, et al., Cell Immunol. 1998; 186(1): 18-27; Allison A C; Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418). Toll like receptors (TLRs) may also be used as adjuvants, and are important members of the family of pattern recognition receptors (PRRs) which recognize conserved motifs shared by many micro-organisms, termed “pathogen-associated molecular patterns” (PAMPS).

In some embodiments, the adjuvant is a dendritic cell targeting molecule (DC). DC is potent and is responsible for initiating antigen-specific immune responses. One biological feature of DCs is their ability to sense conditions under which antigen is encountered, initiating a process of “DC maturation”. Using receptors for various microbial and inflammatory products, DCs respond to antigen exposure in different ways depending on the nature of the pathogen (virus, bacteria, protozoan) encountered. This information is transmitted to T cells by altered patterns of cytokine release at the time of antigen presentation in lymph nodes, altering the type of T cell response elicited. Thus, targeting DCs provides the opportunity not only to quantitatively enhance the delivery of antigen and antigen responses in general, but to qualitatively control the nature of the immune response depending on the desired vaccination outcome.

Dendritic cells express a number of cell surface receptors that can mediate the endocytosis of bound antigen. Targeting exogenous antigens to internalizing surface molecules on systemically-distributed antigen presenting cells facilitates uptake of antigens and thus overcomes a major rate-limiting step in immunization and thus in vaccination.

Dendritic cell targeting molecules include monoclonal or polyclonal antibodies or fragments thereof that recognize and bind to epitopes displayed on the surface of dendritic cells. Dendritic cell targeting molecules also include ligands which bind to a cell surface receptor on dendritic cells. One such receptor, the lectin DEC-205, has been used in vitro and in mice to boost both humoral (antibody-based) and cellular (CD8 T cell) responses by 2-4 orders of magnitude (see, e.g., Hawiger, et al., J. Exp. Med., 194(6):769-79 (2001); Bonifaz, et al., J. Exp. Med., 196(12):1627-38 (2002); Bonifaz, et al., J. Exp. Med., 199(6):815-24 (2004)).

A variety of other endocytic receptors, including a mannose-specific lectin (mannose receptor) and IgG Fc receptors, have also been targeted in this way with similar enhancement of antigen presentation efficiency. Other suitable receptors which may be targeted include, but are not limited to, DC-SIGN, 33D1, SIGLEC-H, DCIR, CD11c, heat shock protein receptors and scavenger receptors.

In some embodiments, the adjuvant is CpG. CpG immuno stimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of Th1 cells and strong cytotoxic T-lymphocyte (CTL) generation, even in the absence of CD4 T-cell help. The Th1 bias induced by TLR9 stimulation is maintained even in the presence of vaccine adjuvants such as alum or incomplete Freund's adjuvant (IFA) that normally promote a Th2 bias. CpG oligonucleotides show even greater adjuvant activity when formulated or co-administered with other adjuvants or in formulations such as microparticles, nano particles, lipid emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5, Jun. 2006, 471-484). U.S. Pat. No. 6,406,705 B1 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which is a preferred component of the pharmaceutical composition of the present invention. Other TLR binding molecules such as RNA binding TLR 7, TLR 8 and/or TLR 9 may also be used.

Xanthenone derivatives such as, for example, Vadimezan or AsA404 (also known as 5,6-dimethylaxanthenone-4-acetic acid (DMXAA)), may also be used as adjuvants according to embodiments of the invention. Alternatively, such derivatives may also be administered in parallel to the vaccine of the invention, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5, 6-Dimethylxanthenone-4-Acetic Acid, Journal of Immunology, 190:5216-25 and Kim et al. (2013) Anticancer Flavonoids are Mouse-Selective STING Agonists, 8: 1396-1401). Other examples of useful adjuvants include, but are not limited to, chemically modified CpGs (e.g. CpR, Idera), Poly(I:C)(e.g. poly:C12U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

Poly-ICLC is a synthetically prepared double-stranded RNA consisting of polyl and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′ 5′-OAS and the P1/eIF2a kinase, also known as the PKR (4-6), as well as RIG-I helicase and MDA5.

In some embodiments, the antigen and adjust are conjugated to outer surface of the composition. In some embodiments, the composition is synthesized with thiol-reactive phospholipids that permit reduction-sensitive linkage of the antigen and/or adjuvant. In some embodiments, loading of the DC within the composition is facilitated through cholesterol modification of the DC molecule. In some embodiments, lyophilization methods are used for the preparation of homogenous compositions. In some embodiments, phospholipids and ApoA mimetic peptides are dissolved in glacial acetic acid and lyophilized. In some embodiments, antigen peptides are incubated with the composition in a buffer (e.g., a sodium phosphate buffer (pH 7.4)) (e.g., at room temperature for 3 hours) to allow for the conjugation of antigen peptides. In some embodiments, the unconjugated antigen peptides are removed using a desalting column (MWCO=7000 Da).

In some embodiments, the adjuvant is conjugated with a hydrophobic molecule. In some embodiments, the average size of the nanoparticle is between 6 to 500 nm.

In some embodiments, the hydrophobic molecule is a lipid molecule. In some embodiments, the lipid molecule is a membrane-forming lipid molecule. In some embodiments, the lipid molecule molecule is a non-membrane-forming lipid molecule.

Examples of lipid molecules applicable with the embodiments of the present invention include, but are not limited to, phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄carbon chains, e.g., lauroyl, myristoyl, palmitoyl, stearoyl, or oleoyl.

Other non-limiting examples of lipid molecules include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof.

Other examples of lipid molecules suitable for use in the present invention include nonphosphorous containing lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

Other examples of lipid molecules suitable for use in the present invention include fatty acids and derivatives or analogs thereof. They include oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p. 92: Muranishi, Critical Reviews in Therapeutic Drug Carrier Systems. 1990, 1-33; El Hariri et al., Pharm. Pharmacol., 1992, 44, 651-654).

Other examples of lipid molecules suitable for use in the present invention include a lipid molecule modified with PEG (PEG-lipid). Examples of PEG-lipids include, but are not limited to, PEG coupled to dialkyloxypropyls (PEG-DAA) as described in, e.g., PCT Publication No. WO 05/026372, PEG coupled to diacylglycerol (PEG-DAG) as described in, e.g., U.S. Patent Publication Nos. 20030077829 and 2005008689, PEG coupled to phospholipids such as phosphatidylethanolamine (PEG-PE), PEG conjugated to ceramides as described in, e.g., U.S. Pat. No. 5,885,613, PEG conjugated to cholesterol or a derivative thereof, and mixtures thereof. The disclosures of these patent documents are herein incorporated by reference in their entirety for all purposes. Additional PEG-lipids include, without limitation, PEG-C-DOMG, 2 KPEG-DMG, and a mixture thereof.

In certain embodiments, the compositions are further associated with any kind of biomacromolecule agent.

In some embodiments, the biomacromolecule agent is a peptide.

For example, in some embodiments, the peptide is an antigen.

In some embodiments, the antigen is a tumor antigen. The antigen can be a tumor antigen, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pm1-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C₂, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGS), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), human EGFR protein or its fragments, such as human EGFR residues 306-325 (SCVRACGADSYEMEEDGVRK (SEQ ID NO:374)) and residues 897-915 (VWSYGVTVWELMTFGSKPY (SEQ ID NO: 13)), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, TPS, WT1 (and WT1-derivaed peptide sequences: WT1 126-134 (RMFP NAPYL (SEQ ID NO:376)), WT1 122-140 (SGQARMFPNAPYLPSCLES (SEQ ID NO: 15)), and WT1 122-144 (SGQARMFPNAPYLPSCLESQPTI (SEQ ID NO: 16)), MUC1 (and MUC1-derived peptides and glycopeptides such as RPAPGS (SEQ ID NO: 17), PPAHGVT (SEQ ID NO: 18), and PDTRP (SEQ ID NO: 19))), LMP2, EGFRvIII, Idiotype, GD2, Ras mutant, p53 mutant, Proteinase3 (PR1), Survivin, hTERT, Sarcoma translocation breakpoints, EphA2, EphA4, LMW-PTP, PAP, ML-IAP, AFP, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, sLe(animal), CYP1B1, PLAC1, GM3, BORIS, Tn, GloboH, NY-BR-1, RGS5, SART3, STn, Carbonic anhydrase IX, PAX5, OY-TES1, Sperm protein 17, LCK, HMWMAA, AKAP-4, XAGE 1, B7H3, Legumain, Tie 2, Page4, VEGFR2, MAD-CT-1, FAP, PDGFR-alpha, PDGFR-β, MAD-CT-2, Fos-related antigen 1, ERBB2, Folate receptor 1 (FOLR1 or FBP), IDH1, IDO, LY6K, fms-related tyro-sine kinase 1 (FLT1, best known as VEGFR1), KDR, PADRE, TA-CIN (recombinant HPV16 L2E7E6), SOX2, and aldehyde dehydrogenase.

In some embodiments wherein the biomacromolecule is an antigen, the composition further comprises an adjuvant.

Such embodiments are not limited to particular antigen. Indeed, antigens can be peptides, proteins, polysaccharides, saccharides, lipids, glycolipids, nucleic acids, or combinations thereof. The antigen can be derived from any source, including, but not limited to, a virus, bacterium, parasite, plant, protozoan, fungus, tissue or transformed cell such as a cancer or leukemic cell and can be a whole cell or immunogenic component thereof, e.g., cell wall components or molecular components thereof.

In some embodiments, the antigens are known in the art and are available from commercial government and scientific sources. In some embodiments, the antigens are whole inactivated or attenuated organisms. These organisms may be infectious organisms, such as viruses, parasites and bacteria. These organisms may also be tumor cells. The antigens may be purified or partially purified polypeptides derived from tumors or viral or bacterial sources. Criteria for identifying and selecting effective antigenic peptides (e.g., minimal peptide sequences capable of eliciting an immune response) can be found in the art. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

In some embodiments, the antigen is a self antigen. As used herein, the term “self-antigen” refers to an immunogenic antigen or epitope which is native to a mammal and which may be involved in the pathogenesis of an autoimmune disease.

In some embodiments, the antigen is a viral antigen. Viral antigens can be isolated from any virus including, but not limited to, a virus from any of the following viral families: Arenaviridae, Arterivirus, Astroviridae, Baculoviridae, Badnavirus, Barnaviridae, Bimaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

In some embodiments, the antigen is a bacterial antigen. Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

In some embodiments, the antigen is a parasite antigen. Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

In some embodiments, the antigen is an allergen and environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula), alder (Alnus), hazel (Corylus), hornbeam (Carpinus) and olive (Olea), cedar (Cryptomeria and Juniperus), Plane tree (Platanus), the order of Poales including i.e. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

In some embodiments, the antigen is a tumor antigen (described herein).

One of the critical barriers to developing curative and tumor-specific immunotherapy is the identification and selection of highly specific and restricted tumor antigens to avoid autoimmunity. Tumor neo-antigens, which arise as a result of genetic change (e.g., inversions, translocations, deletions, missense mutations, splice site mutations, etc.) within malignant cells, represent the most tumor-specific class of antigens.

In some embodiments, the antigen is a neo-antigen. The term neoantigen is used herein to define any newly expressed antigenic determinant. Neoantigens may arise upon conformational change in a protein, as newly expressed determinants (especially on the surfaces of transformed or infected cells), as the result of complex formation of one or more molecules or as the result of cleavage of a molecule with a resultant display of new antigenic determinants. Thus, as used herein, the term neoantigen covers antigens expressed upon infection (e.g. viral infection, protozoal infection or bacterial infection), in prion-mediated diseases, an on cell transformation (cancer), in which latter case the neoantigen may be termed a tumour-associated antigen.

The present invention is not limited to a particular manner of identifying neo-antigens. In some embodiments, identification of neo-antigens involves identifying all, or nearly all, mutations in the neoplasia/tumor at the DNA level using whole genome sequencing, whole exome (e.g., only captured exons) sequencing, or RNA sequencing of tumor versus matched germline samples from each patient. In some embodiments, identification of neo-antigens involves analyzing the identified mutations with one or more peptide-MHC binding prediction algorithms to generate a plurality of candidate neo-antigen T cell epitopes that are expressed within the neoplasia/tumor and may bind patient HLA alleles. In some embodiments, identification of neo-antigens involves synthesizing the plurality of candidate neo-antigen peptides selected from the sets of all neo open reading frame peptides and predicted binding peptides for use in a cancer vaccine.

As such, the present invention is based, at least in part, on the ability to identify all, or nearly all, of the mutations within a neoplasia/tumor (e.g., translocations, inversions, large and small deletions and insertions, missense mutations, splice site mutations, etc.). In particular, these mutations are present in the genome of neoplasia/tumor cells of a subject, but not in normal tissue from the subject. Such mutations are of particular interest if they lead to changes that result in a protein with an altered amino acid sequence that is unique to the patient's neoplasia/tumor (e.g., a neo-antigen). For example, useful mutations may include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift mutations or deletions that lead to a new open reading frame with a novel tumor-specific protein sequence; and the like. Peptides with mutations or mutated polypeptides arising from, for example, splice-site, frameshift, read-through, or gene fusion mutations in tumor cells may be identified by sequencing DNA, RNA or protein in tumor versus normal cells.

Also within the scope of the present invention is personal neo-antigen peptides derived from common tumor driver genes and may further include previously identified tumor specific mutations.

Preferably, any suitable sequencing-by-synthesis platform can be used to identify mutations. Four major sequencing-by-synthesis platforms are currently available: the Genome Sequencers from Roche/454 Life Sciences, the HiSeq Analyzer from Illumina/Solexa, the SOLiD system from Applied BioSystems, and the Heliscope system from Helicos Biosciences. Sequencing-by-synthesis platforms have also been described by Pacific Biosciences and VisiGen Biotechnologies. Each of these platforms can be used in the methods of the invention. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair. Subsequent to the capture, the sequence may be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or in a step-and-repeat mode. For real-time analysis, different optical labels to each nucleotide may be incorporated and multiple lasers may be utilized for stimulation of incorporated nucleotides.

Any cell type or tissue may be utilized to obtain nucleic acid samples for use in the sequencing methods described herein. In some embodiments, the DNA or RNA sample is obtained from a neoplasia/tumor or a bodily fluid, e.g., blood, obtained by known techniques (e.g. venipuncture) or saliva. Alternatively, nucleic acid tests can be performed on dry samples (e.g. hair or skin).

A variety of methods are available for detecting the presence of a particular mutation or allele in an individual's DNA or RNA. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

PCR based detection means may include multiplex amplification of a plurality of markers simultaneously. For example, it is well known in the art to select PCR primers to generate PCR products that do not overlap in size and can be analyzed simultaneously.

Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. In one embodiment, the single base polymorphism can be detected by using a specialized exonuclease-resistant nucleotide, as disclosed, e.g., U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (see, e.g, French Patent No. 2,650,840; PCT Application No. WO1991/02087). As in the method of U.S. Pat. No. 4,656,127, a primer may be employed that is complementary to allelic sequences immediately 3′ to a polymorphic site.

The method determines the identity of the nucleotide of that site using labeled dideoxynucleotide derivatives, which, if complementary to the nucleotide of the polymorphic site, will become incorporated onto the terminus of the primer.

An alternative method, known as Genetic Bit Analysis or GBA® is described in PCT Application No. WO 1992/15712). GBA® uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO1991/02087) the GBA® method is preferably a heterogeneous phase assay, in which the primer or the target molecule is immobilized to a solid phase. Recently, several primer-guided nucleotide incorporation procedures for assaying polymorphic sites in DN A have been described (see, e.g., Komher, J. S. et al., Nucl. Acids. Res. 17:7779-7784 (1989); Sokolov, B. P., Nucl. Acids Res. 18:3671 (1990); Syvanen, A.-C, et al., Genomics 8:684-692 (1990); Kuppuswamy, M. N. et al., Proc. Natl. Acad. Sci. (U.S.A.) 88: 1143-1147 (1991); Prezant, T. R. et al., Hum. Mutat. 1: 159-164 (1992); Ugozzoli, L. et al., GATA 9: 107-112 (1992); Nyren, P. et al., Anal. Biochem. 208: 171-175 (1993)). These methods differ from GBA® in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (see, e.g., Syvanen, A.-C, et al., Amer. J. Hum. Genet. 52:46-59 (1993)).

An alternative method for identifying tumor specific neo-antigens is direct protein sequencing. Protein sequencing of enzymatic digests using multidimensional MS techniques (MSn) including tandem mass spectrometry (MS/MS)) can also be used to identify neo-antigens of the invention. Such proteomic approaches permit rapid, highly automated analysis (see, e.g., K. Gevaert and J. Vandekerckhove, Electrophoresis 21: 1145-1154 (2000)). It is further contemplated within the scope of the invention that high-throughput methods for de novo sequencing of unknown proteins may be used to analyze the proteome of a patient's tumor to identify expressed neo-antigens. For example, meta shotgun protein sequencing may be used to identify expressed neo-antigens (see, e.g., Guthals et al. (2012) Shotgun Protein Sequencing with Meta-contig Assembly, Molecular and Cellular Proteomics 11(10): 1084-96).

Tumor specific neo-antigens may also be identified using MHC multimers to identify neo-antigen-specific T-cell responses. For example, highthroughput analysis of neo-antigen-specific T-cell responses in patient samples may be performed using MHC tetramer-based screening techniques (see, e.g., Hombrink et al. (2011) High-Throughput Identification of Potential Minor Histocompatibility Antigens by MHC Tetramer-Based Screening: Feasibility and Limitations 6(8): 1-11; Hadrup et al. (2009) Parallel detection of antigen-specific T-cell responses by multidimensional encoding of MHC multimers, Nature Methods, 6(7):520-26; van Rooij et al. (2013) Tumor exome analysis reveals neoantigen-specific T-cell reactivity in an Ipilimumab-responsive melanoma, Journal of Clinical Oncology, 31: 1-4; and Heemskerk et al. (2013) The cancer antigenome, EMBO Journal, 32(2): 194-203). It is contemplated within the scope of the invention that such tetramer-based screening techniques may be used for the initial identification of tumor specific neo-antigens, or alternatively as a secondary screening protocol to assess what neo-antigens a patient may have already been exposed to, thereby facilitating the selection of candidate neo-antigens for the vaccines of the invention.

The invention further includes isolated peptides (e.g., neo-antigenic peptides containing the tumor specific mutations identified by the described methods, peptides that comprise known tumor specific mutations, and mutant polypeptides or fragments thereof identified by the described methods). These peptides and polypeptides are referred to herein as “neo-antigenic peptides” or “neo-antigenic polypeptides.” The polypeptides or peptides can be of a variety of lengths and will minimally include the small region predicted to bind to the HLA molecule of the patient (the “epitope”) as well as additional adjacent amino acids extending in both the N- and C-terminal directions. The polypeptides or peptides can be either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

In certain embodiments the size of the at least one neo-antigenic peptide molecule may comprise, but is not limited to, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the neo-antigenic peptide molecules are equal to or less than 50 amino acids. In a preferred embodiment, the neo-antigenic peptide molecules are equal to about 20 to about 30 amino acids.

In some embodiments, the peptide is Adrenocorticotropic Hormone (ACTH), a growth hormone peptide, a Melanocyte Stimulating Hormone (MSH), Oxytocin, Vasopressin, Corticotropin Releasing Factor (CRF), a CRF-related peptide, a Gonadotropin Releasing Hormone Associated Peptide (GAP), Growth Hormone Releasing Factor (GRF), Lutenizing Hormone Release Hormone (LH-RH), an orexin, a Prolactin Releasing Peptide (PRP), a somatostatin, Thyrotropin Releasing Hormone (THR), a THR analog, Calcitonin (CT), a CT-precursor peptide, a Calcitonin Gene Related Peptide (CGRP), a Parathyroid Hormone (PTH), a Parathyroid Hormone Related Protein (PTHrP), Amylin, Glucagon, Insulin, an Insulin-like peptide, NeuroPeptide Y (NPY), a Pancreatic Polypeptide (PP), Peptide YY (PYY), Cholecystokinin (CCK), a CCK-related peptide, Gastrin Releasing Peptide (GRP), Gastrin, a Gastrin-related peptide, a Gastrin inhibitory peptide, Motilin, Secretin, Vasoactive Intestinal Peptide (VIP), a VIP-related peptide, an Atrial-Natriuretic Peptide (ANP), a Brain Natriuretic Peptide (BNP), a C-Type Natriuretic Peptide(CNP), a tachykinin, an angiotensin, a renin substrate, a renin inhibitor, an endothelin, an endothelin-related peptide, an opioid peptide, a thymic peptide, an adrenomedullin peptide, an allostatin peptide, an amyloid beta-protein fragment, an antimicrobial peptide, an antioxidant peptide, an apoptosis related peptide, a Bag Cell Peptide (BCPs), Bombesin, a bone Gla protein peptide, a Cocaine and Amphetamine Related Transcript (CART) peptide, a cell adhesion peptide, a chemotactic peptide, a complement inhibitor, a cortistatin peptide, a fibronectin fragment, a fibrin related peptide, FMRF, a FMRF amide-related peptide (FaRP), Galanin, a Galanin-related peptide, a growth factor, a growth factor-related peptide, a G-Therapeutic Peptide-Binding Protein fragment, Gualylin, Uroguanylin, an Inhibin peptide, Interleukin (IL), an Interleukin Receptor protein, a laminin fragment, a leptin fragment peptide, a leucokinin, Pituitary Adenylate Cyclase Activating Polypeptide (PAPCAP), Pancreastatin, a polypeptide repetitive chain, a signal transducing reagent, a thrombin inhibitor, a toxin, a trypsin inhibitor, a virus-related peptide, an adjuvant peptide analog, Alpha Mating Factor, Antiarrhythmic Peptide, Anorexigenic Peptide, Alpha-1 Antitrypsin, Bovine Pineal Antireproductive Peptide, Bursin, C3 Peptide P16, Cadherin Peptide, Chromogranin A Fragment, Contraceptive Tetrapeptide, Conantokin G, Conantokin T, Crustacean Cardioactive Peptide, C-Telopeptide, Cytochrome b588 Peptide, Decorsin, Delicious Peptide, Delta-Sleep-Inducing Peptide, Diazempam-Binding Inhibitor Fragment, Nitric Oxide Synthase Blocking Peptide, OVA Peptide, Platelet Calpain Inhibitor (P1), Plasminogen Activator Inhibitor 1, Rigin, Schizophrenia Related Peptide, Sodium Potassium Atherapeutic Peptidase Inhibitor-1, Speract, Sperm Activating Peptide, Systemin, a Thrombin receptor agonist, Tuftsin, Adipokinetic Hormone, Uremic Pentapeptide, Antifreeze Polypeptide, Tumor Necrosis Factor (TNF), Leech [Des Asp10]Decorsin, L-Ornithyltaurine Hydrochloride, P-Aminophenylacetyl Tuftsin, Ac-Glu-Glu-Val-Val-Ala-Cys-pNA (SEQ ID NO: 1), Ac-Ser-Asp-Lys-Pro (SEQ ID NO: 2), Ac-rfwink-NH2, Cys-Gly-Tyr-Gly-Pro-Lys-Lys-Lys-Arg-Lys-Val-Gly-Gly (SEQ ID NO: 3), D-Ala-Leu, D-D-D-D-D (SEQ ID NO: 4), D-D-D-D-D-D (SEQ ID NO: 5), N-P-N-A-N-P-N-A (SEQ ID NO: 6), V-A-I-T-V-L-V-K (SEQ ID NO: 7), V-G-V-R-V-R (SEQ ID NO: 8), V-I-H-S(SEQ ID NO: 9), V-P-D-P-R (SEQ ID NO: 10), Val-Thr-Cys-Gly (SEQ ID NO: 11), Sea Urchin Sperm Activating Peptide, a SHU-9119 antagonist, a MC3-R antagonist, a MC4-R antagonist, Glaspimod, HP-228, Alpha 2-Plasmin Inhibitor, APC Tumor Suppressor, Early Pregnancy Factor, Gamma Interferon, Glandular Kallikrei N-1, Placental Ribonuclease Inhibitor, Sarcolecin Binding Protein, Surfactant Protein D, Wilms' Tumor Suppressor, GABAB 1b Receptor Peptide, Prion Related Peptide (iPRP13), Choline Binding Protein Fragment, Telomerase Inhibitor, Cardiostatin Peptide, Endostatin Derived Peptide, Prion Inhibiting Peptide, N-Methyl D-Aspartate Receptor Antagonist, and C-PeptideAnalog.

In some embodiments, the peptide is selected from 177Lu-DOTA0-Tyr3—Octreotate, Abarelix acetate, ADH-1, Afamelanotidec, melanotan-1, CUV1647, Albiglutide, Aprotinin, Argipressin, Atosiban acetate, Bacitracin, Bentiromide, a BH3 domain, Bivalirudin, Bivalirudin trifluoroacetate hydrate, Blisibimod, Bortezomib, Buserelin, Buserelin acetate, Calcitonin, Carbetocin, Carbetocin acetate, Cecropin A and B, Ceruletide, Ceruletide diethylamine, Cetrorelix, Cetrorelix acetate, Ciclosporine, Cilengitidec, EMD121974, Corticorelin acetate injection, hCRF, Corticorelin ovine triflutate, corticorelin trifluoroacetate, Corticotropin, Cosyntropin, ACTH 1-24, tetracosactide hexaacetate, Dalbavancin, Daptomycin, Degarelix acetate, Depreotide trifluoroacetate (plus sodium pertechnetate), Desmopressin acetate, Desmopressin DDAVP, Dulaglutide, Ecallantide, Edotreotide (plus yttrium-90), Elcatonin acetate, Enalapril maleate (or 2-butanedioate), Enfuvirtide, Eptifibatide, Exenatide, Ganirelix acetate, Glatiramer acetate, Glutathion, Gonadorelin, Gonadorelin acetate, GnRH, LHRH, Goserelin, Goserelin acetate, Gramicidin, Histrelin acetate, Human calcitonin, Icatibant, Icatibant acetate, IM862, oglufanide disodium, KLAKLAK, Lanreotide acetate, Lepirudin, Leuprolide, Leuprolide acetate, leuprorelin, Liraglutide, Lisinopril, Lixisenatide, Lypressin, Magainin2, MALP-2Sc, macrophage-activating lipopeptide-2 synthetic, Nafarelin acetate, Nesiritide, NGR-hTNF, Octreotide acetate, Oritavancin, Oxytocin, Pasireotide, Peginesatide, Pentagastrin, Pentetreotide (plus indium-111), Phenypressin, Pleurocidin, Pramlintide, Protirelin, thyroliberin, TRH, TRF, Salmon calcitonin, Saralasin acetate, Secretin (human), Secretin (porcine), Semaglutide, Seractide acetate, ACTH, corticotropin, Sermorelin acetate, GRF 1-29, Sinapultide, KL4 in lucinactant, Sincalide, Somatorelin acetate, GHRH, GHRF, GRF, Somatostatin acetate, Spaglumat magnesium (or sodium) salt, Substance P, Taltirelin hydrate, Teduglutide, Teicoplanin, Telavancin, Teriparatide, Terlipressin acetate, Tetracosactide, Thymalfasin, thymosin a-1, Thymopentin, Trebananib, Triptorelin, Triptorelin pamoate, Tyroserleutide, Ularitide, Vancomycin, Vapreotide acetate, Vasoactive intestinal peptide acetate, Vx-001c, TERT572Y, Ziconotide acetate, α5-α6 Bax peptide, and β-defensin.

In some embodiments, the peptide is any peptide which would assist in achieving a desired purpose with the composition. For example, in some embodiments, the peptide is any peptide that will facilitate treatment of any type of disease and/or disorder.

In some embodiments, the biomacromolecule agent is a nucleic acid. Such embodiments encompass any type of nucleic acid molecule including, but not limited to, RNA, siRNA, microRNA, interference RNA, mRNA, replicon mRNA, RNA-analogues, and DNA.

As such, the present invention provides such compositions associated with one or more neo-antigenic peptides. In some embodiments, the composition is associated with two neo-antigenic peptides. In some embodiments, the composition is associated with at least 5 or more neo-antigenic peptides. In some embodiments, the composition is associated with at least about 6, about 8, about 10, about 12, about 14, about 16, about 18, or about 20 distinct peptides. In some embodiments, the composition is associated with at least 20 distinct peptides.

The neo-antigenic peptides, polypeptides, and analogs can be further modified to contain additional chemical moieties not normally part of the protein. Those derivatized moieties can improve the solubility, the biological half-life, absorption of the protein, or binding affinity. The moieties can also reduce or eliminate any desirable side effects of the proteins and the like. An overview for those moieties can be found in Remington's Pharmaceutical Sciences, 20^{th} ed., Mack Publishing Co., Easton, PA (2000). For example, neo-antigenic peptides and polypeptides having the desired activity may be modified as necessary to provide certain desired attributes, e.g. improved pharmacological characteristics, while increasing or at least retaining substantially all of the biological activity of the unmodified peptide to bind the desired MHC molecule and activate the appropriate T cell. For instance, the neo-antigenic peptide and polypeptides may be subject to various changes, such as substitutions, either conservative or non-conservative, where such changes might provide for certain advantages in their use, such as improved MHC binding. Such conservative substitutions may encompass replacing an amino acid residue with another amino acid residue that is biologically and/or chemically similar, e.g., one hydrophobic residue for another, or one polar residue for another. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, III., Pierce), 2d Ed. (1984).

In some embodiments, the neo-antigenic peptides and polypeptides may be modified with linking agents for purposes of facilitating association with composition. The invention is not limited to a particular type or kind of linking agent. In some embodiments, the linking agent is a cysteine-serine-serine (CSS) molecule.

The neo-antigenic peptide and polypeptides may also be modified by extending or decreasing the compound's amino acid sequence, e.g., by the addition or deletion of amino acids. The neo-antigenic peptides, polypeptides, or analogs can also be modified by altering the order or composition of certain residues. It will be appreciated by the skilled artisan that certain amino acid residues essential for biological activity, e.g., those at critical contact sites or conserved residues, may generally not be altered without an adverse effect on biological activity. The non-critical amino acids need not be limited to those naturally occurring in proteins, such as L-a-amino acids, or their D-isomers, but may include non-natural amino acids as well, such as β-γ-δ-amino acids, as well as many derivatives of L-a-amino acids.

Typically, a neo-antigen polypeptide or peptide may be optimized by using a series of peptides with single amino acid substitutions to determine the effect of electrostatic charge, hydrophobicity, etc. on MHC binding. For instance, a series of positively charged (e.g., Lys or Arg) or negatively charged (e.g., Glu) amino acid substitutions may be made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding. Amino acid substitutions are typically of single residues. Substitutions, deletions, insertions or any combination thereof may be combined to arrive at a final peptide.

One of skill in the art will appreciate that there are a variety of ways in which to produce such tumor specific neo-antigens. In general, such tumor specific neo-antigens may be produced either in vitro or in vivo. Tumor specific neo-antigens may be produced in vitro as peptides or polypeptides, which may then be formulated into a personalized neoplasia vaccine and administered to a subject. Such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide.

Alternatively, tumor specific neo-antigens may be produced in vivo by introducing molecules (e.g., DNA, RNA, viral expression systems, and the like) that encode tumor specific neo-antigens into a subject, whereupon the encoded tumor specific neo-antigens are expressed.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963).

A further aspect of the invention provides a nucleic acid (e.g., a polynucleotide) encoding a neo-antigenic peptide of the invention, which may be used to produce the neo-antigenic peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. A still further aspect of the invention provides an expression vector capable of expressing a polypeptide according to the invention. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

The invention further embraces variants and equivalents which are substantially homologous to the identified tumor specific neo-antigens described herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid. What is intended by a conservative amino acid substitution is well known in the art.

The invention also includes expression vectors comprising the isolated polynucleotides, as well as host cells containing the expression vectors. It is also contemplated within the scope of the invention that the neo-antigenic peptides may be provided in the form of RNA or cDNA molecules encoding the desired neo-antigenic peptides. The invention also provides that the one or more neo-antigenic peptides of the invention may be encoded by a single expression vector. The invention also provides that the one or more neo-antigenic peptides of the invention may be encoded and expressed in vivo using a viral based system (e.g., an adenovirus system).

The term “polynucleotide encoding a polypeptide” encompasses a polynucleotide which includes only coding sequences for the polypeptide as well as a polynucleotide which includes additional coding and/or non-coding sequences. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides may comprise the coding sequence for the tumor specific neo-antigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In some embodiments, the polynucleotides can comprise the coding sequence for the tumor specific neo-antigenic peptide fused in the same reading frame to a marker sequence that allows, for example, for purification of the encoded polypeptide, which may then be incorporated into the personalized neoplasia vaccine. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like. In embodiments, the polynucleotides may comprise the coding sequence for one or more of the tumor specific neo-antigenic peptides fused in the same reading frame to create a single concatamerized neo-antigenic peptide construct capable of producing multiple neo-antigenic peptides.

In embodiments, the present invention provides isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tumor specific neo-antigenic peptide of the present invention.

By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, WI 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

The isolated tumor specific neo-antigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585.

In embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest will be inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host. Recombinant expression vectors may be used to amplify and express DNA encoding the tumor specific neo-antigenic peptides. Recombinant expression vectors are replicable DNA constructs which have synthetic or cDNA-derived DNA fragments encoding a tumor specific neo-antigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transforaiants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23: 175, 1981), and other cell lines capable of expressing an appropriate vector including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein. Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

As such, in certain embodiments, the present invention relates to personalized strategies for the treatment of disorders (e.g., neoplasia), and more particularly tumors, by administering a therapeutically effective amount of a composition (as described herein) and one or more neoplasia/tumor specific neo-antigens to a subject (e.g., a mammal such as a human) (e.g., a vaccine composition capable of raising a specific T-cell response). Indeed, in certain embodiments, whole genome/ex ome sequencing may be used to identify all, or nearly all, mutated neo-antigens that are uniquely present in a neoplasia/tumor of an individual patient, and that this collection of mutated neo-antigens may be analyzed to identify a specific, optimized subset of neo-antigens for use as a personalized cancer vaccine for treatment of the patient's neoplasia/tumor. For example, in some embodiments, a population of neoplasia/tumor specific neo-antigens may be identified by sequencing the neoplasia/tumor and normal DNA of each patient to identify tumor-specific mutations, and determining the patient's HLA allotype. The population of neoplasia/tumor specific neo-antigens and their cognate native antigens may then be subject to bioinformatic analysis using validated algorithms to predict which tumor-specific mutations create epitopes that could bind to the patient's HLA allotype, and in particular which tumor-specific mutations create epitopes that could bind to the patient's HLA allotype more effectively than the cognate native antigen. Based on this analysis, one or more peptides corresponding to a subset of these mutations may be designed and synthesized for each patient, and pooled together for use as a cancer vaccine in immunizing the patient. The neo-antigens peptides may be combined another anti-neoplastic agent. In some embodiments, such neo-antigens are expected to bypass central thymic tolerance (thus allowing stronger antitumor T cell response), while reducing the potential for autoimmunity (e.g., by avoiding targeting of normal self-antigens).

The invention further provides a method of inducing a neoplasia/tumor specific immune response in a subject, vaccinating against a neoplasia/tumor, treating and or alleviating a symptom of cancer in a subject by administering the subject a neo-antigenic peptide or vaccine composition of the invention.

According to the invention, the above-described cancer vaccine may be used for a patient that has been diagnosed as having cancer, or at risk of developing cancer. In one embodiment, the patient may have a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

The peptide or composition of the invention is administered in an amount sufficient to induce a CTL response. The neo-antigenic peptide, polypeptide or vaccine composition of the invention can be administered alone or in combination with other therapeutic agents. The therapeutic agent is for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. For prostate cancer treatment, a preferred chemotherapeutic agent with which anti-CTLA-4 can be combined is paclitaxel (Taxol®).

In addition, the subject may be further administered an anti-immunosuppressive or immuno stimulatory agent. For example, the subject is further administered an anti-CTLA-4 antibody, anti-PD-1, anti-PD-LI, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, and inhibitors of IDO. Blockade of CTLA-4 or PD-1/PD-L1 by antibodies can enhance the immune response to cancerous cells in the patient. In particular, CTLA-4 blockade has been shown effective when following a vaccination protocol.

The optimum amount of each peptide to be included in the vaccine composition and the optimum dosing regimen can be determined by one skilled in the art without undue experimentation. For example, the peptide or its variant may be prepared for intravenous (i.v.) injection, sub-cutaneous (s.c.) injection, intradermal (i.d.) injection, intraperitoneal (i.p.) injection, intramuscular (i.m.) injection. Preferred methods of peptide injection include s.c, i.d., i.p., i.m., and i.v. Preferred methods of DNA injection include i.d., i.m., s.c, i.p. and i.v. For example, doses of between 1 and 500 mg 50 μg and 1.5 mg, preferably 10 μg to 500 μg, of peptide or DNA may be given and will depend from the respective peptide or DNA. Doses of this range were successfully used in previous trials (Brunsvig P F, et al., Cancer Immunol Immunother. 2006; 55(12): 1553-1564; M. Staehler, et al., ASCO meeting 2007; Abstract No 3017). Other methods of administration of the vaccine composition are known to those skilled in the art.

The inventive vaccine may be compiled so that the selection, number and/or amount of peptides present in the composition is/are tissue, cancer, and/or patient-specific. For instance, the exact selection of peptides can be guided by expression patterns of the parent proteins in a given tissue to avoid side effects. The selection may be dependent on the specific type of cancer, the status of the disease, earlier treatment regimens, the immune status of the patient, and, of course, the HLA-haplotype of the patient. Furthermore, the vaccine according to the invention can contain individualized components, according to personal needs of the particular patient. Examples include varying the amounts of peptides according to the expression of the related neoantigen in the particular patient, unwanted side-effects due to personal allergies or other treatments, and adjustments for secondary treatments following a first round or scheme of treatment.

Such vaccines may be administered to an individual already suffering from cancer. In therapeutic applications, such vaccines are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition and possibly by measuring specific CTL activity in the patient's blood. It should be kept in mind that the peptide and compositions of the present invention may generally be employed in serious disease states, that is, life-threatening or potentially life threatening situations, especially when the cancer has metastasized. For therapeutic use, administration should begin as soon as possible after the detection or surgical removal of tumors. This is followed by boosting doses until at least symptoms are substantially abated and for a period thereafter. The pharmaceutical compositions (e.g., vaccine compositions) for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor.

Such embodiments are not limited to a particular manner of characterizing a composition associated with antigen and DC. In some embodiments, the morphology of composition is observed by TEM. In some embodiments, the size distribution of the composition is analyzed by dynamic light scattering (DLS) using a Malven Nanosizer instrument and GPC assay.

The compositions configured to activate an immune response are useful for activating T cells in subjects for prophylactic and therapeutic applications. Activation of T cells by nanoparticle vaccine compositions increases their proliferation, cytokine production, differentiation, effector functions and/or survival. Methods for measuring these are well known to those in the art. The T cells activated by the nanoparticle vaccine compositions can be any cell which express the T cell receptor, including α/β and γ/δ T cell receptors. T-cells include all cells which express CD3, including T-cell subsets which also express CD4 and CD8. T-cells include both naive and memory cells and effector cells such as CTL. T-cells also include regulatory cells such as Th1, Tc1, Th2, Tc2, Th3, Treg, and Tr1 cells. T-cells also include NKT-cells and similar unique classes of the T-cell lineage. In some embodiments, the T cells that are activated are CD8⁺ T cells.

In general, such compositions configured to activate an immune response are useful for treating a subject having or being predisposed to any disease or disorder to which the subject's immune system mounts an immune response. The compositions are useful as prophylactic vaccines, which confer resistance in a subject to subsequent exposure to infectious agents. The compositions are also useful as therapeutic vaccines, which can be used to initiate or enhance a subject's immune response to a pre-existing antigen, such as a tumor antigen in a subject with cancer, or a viral antigen in a subject infected with a virus. The compositions are also useful as desensitizing vaccines, which function to “tolerize” an individual to an environmental antigen, such as an allergen.

The ability to target these compositions to professional antigen-presenting cells such as dendritic cells, and the ability of these compositions to elicit T-cell mediated immune responses by causing cross-presentation of antigens makes these compositions especially useful for eliciting a cell-mediated response to a disease-related antigen in order to attack the disease. Thus, in some embodiments, the type of disease to be treated or prevented is a malignant tumor or a chronic infectious disease caused by a bacterium, virus, protozoan, helminth, or other microbial pathogen that enters intracellularly and is attacked, i.e., by the cytotoxic T lymphocytes.

The desired outcome of a prophylactic, therapeutic or de-sensitized immune response may vary according to the disease, according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease. For example, the stimulation of an immune response against a cancer may be coupled with surgical, chemotherapeutic, radiologic, hormonal and other immunologic approaches in order to affect treatment.

Subjects with or exposed to infectious agents can be treated therapeutically or prophylactically such compositions configured to activate an immune response as disclosed herein. Infectious agents include bacteria, viruses and parasites. In some instances, the subject can be treated prophylactically, such as when there may be a risk of developing disease from an infectious agent. An individual traveling to or living in an area of endemic infectious disease may be considered to be at risk and a candidate for prophylactic vaccination against the particular infectious agent. Preventative treatment can be applied to any number of diseases where there is a known relationship between the particular disease and a particular risk factor, such as geographical location or work environment.

Subjects with or at risk for developing malignant tumors can be treated therapeutically or prophylactically such compositions configured to activate an immune response as disclosed herein. In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site. Such compositions configured to activate an immune response as disclosed herein are useful for treating subjects having malignant tumors.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. A melanoma is a type of carcinoma of the skin for which this invention is particularly useful. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated in with the provided compositions configured to activate an immune response include, but are not limited to, the following: bladder, brain, breast, cervical, colo-rectal, esophageal, kidney, liver, lung, nasopharangeal, pancreatic, prostate, skin, stomach, uterine, and the like. Administration is not limited to the treatment of an existing tumor or infectious disease but can also be used to prevent or lower the risk of developing such diseases in an individual, i.e., for prophylactic use. Potential candidates for prophylactic vaccination include individuals with a high risk of developing cancer, i.e., with a personal or familial history of certain types of cancer.

Subjects with or at risk for exposure to allergens can be treated therapeutically or prophylactically such compositions configured to activate an immune response as disclosed herein. Such compositions may be administered to subjects for the purpose of preventing and/or attenuating allergic reactions, such as allergic reactions which lead to anaphylaxis. Allergic reactions may be characterized by the T_{H}2 responses against an antigen leading to the presence of IgE antibodies. Stimulation of T_{H}1 immune responses and the production of IgG antibodies may alleviate allergic disease. Thus, the compositions configured to activate an immune response as disclosed herein are useful for producing antibodies that prevent and/or attenuate allergic reactions in subjects exposed to allergens.

Subjects with or at risk for immunosuppressed conditions can be treated therapeutically or prophylactically with such compositions configured to activate an immune response as disclosed herein. Such composition-based vaccines disclosed herein can be used for treatment of disease conditions characterized by immunosuppression, including, but not limited to, AIDS or AIDS-related complex, idiopathic immuno suppression, drug induced immunosuppression, other virally or environmentally-induced conditions, and certain congenital immune deficiencies. Such compositions can also be employed to increase immune function that has been impaired by the use of radiotherapy of immunosuppressive drugs (e.g., certain chemotherapeutic agents), and therefore can be particularly useful when used in conjunction with such drugs or radiotherapy.

In general, methods of administering vaccines as disclosed herein (e.g., compositions configured to activate an immune response) are well known in the art. Any acceptable method known to one of ordinary skill in the art may be used to administer a formulation to the subject. The administration may be localized (i.e., to a particular region, physiological system, tissue, organ, or cell type) or systemic. Vaccines can be administered by a number of routes including, but not limited to: oral, inhalation (nasal or pulmonary), intravenous, intraperitoneal, intramuscular, transdermal, subcutaneous, topical, sublingual, or rectal means. Injections can be e.g., intravenous, intradermal, subcutaneous, intramuscular, or intraperitoneal. In some embodiments, the injections can be given at multiple locations.

Administration of the formulations may be accomplished by any acceptable method which allows an effective amount of the vaccine to reach its target. The particular mode selected will depend upon factors such as the particular formulation, the severity of the state of the subject being treated, and the dosage required to induce an effective immune response. As generally used herein, an “effective amount” is that amount which is able to induce an immune response in the treated subject. The actual effective amounts of vaccine can vary according to the specific antigen or combination thereof being utilized, the particular composition formulated, the mode of administration, and the age, weight, condition of the individual being vaccinated, as well as the route of administration and the disease or disorder.

Natural killer T (NKT) cells are a heterogeneous group of T cells that share properties of both T cells and natural killer cells. Many of these cells recognize the non-polymorphic CDld molecule, an antigen-presenting molecule that binds self and foreign lipids and glycolipids. NKT cells constitute only approximately 0.1% of all peripheral blood T cells. NKT cells are a subset of T cells that coexpress an αβ T-cell receptor, but also express a variety of molecular markers that are typically associated with NK cells, such as NK1.1. The best-known NKT cells differ from conventional αβ T cells in that their T-cell receptors are far more limited in diversity (‘invariant’ or ‘type 1’ NKT). They and other CDld-restricted T cells (‘type 2’ NKT) recognize lipids and glycolipids presented by CDld molecules, a member of the CD1 family of antigen-presenting molecules, rather than peptide-major histocompatibility complexes (MHCs). NKT cells include both NK1.1⁺ and NK1.1⁻, as well as CD4⁺, CD4⁻, CD8⁺ and CD8⁻ cells.

In certain embodiments, the compositions comprising agents capable of stimulating an innate immune response in a subject upon administration to the subject are further associated with (e.g., complexed, conjugated, encapsulated, absorbed, adsorbed, admixed) one or more therapeutic agents. Such embodiments are not limited to particular type or kind of therapeutic agent.

In some embodiments, the therapeutic agent configured for treating and/or preventing cancer. Examples of such therapeutic agents include, but are not limited to, chemotherapeutic agents, anti-oncogenic agents, anti-angiogenic agents, tumor suppressor agents, anti-microbial agents, etc.

In some embodiments, the therapeutic agent is configured for treating and/or preventing autoimmune disorders and/or inflammatory disorders. Examples of such therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, parenteral gold or oral gold.

In some embodiments, the therapeutic agent is configured for treating and/or preventing cardiovascular related disorders (e.g., atherosclerosis, heart failure, arrhythmia, atrial fibrillation, hypertension, coronary artery disease, angina pectoris, etc.). Examples of therapeutic agents known to be useful in treating and/or preventing cardiovascular related disorders include, angiotensin-converting enzyme (ACE) inhibitors (e.g., benazepril, enalapril, Lisinopril, perindopril, Ramipril), adenosine, alpha blockers (alpha adrenergic antagonist medications) (e.g., clonidine, guanabenz, labetalol, phenoxybenzamine, terazosin, doxazosin, guanfacine, methyldopa, prazosin), angtiotensin II receptor blockers (ARBs) (e.g., candesartan, irbesartan, olmesartan medoxomil, telmisartan, eprosartan, losartan, tasosartan, valsartan), antiocoagulants (e.g., heparin fondaparinux, warfarin, ardeparin, enoxaparin, reviparin, dalteparin, nadroparin, tinzaparin), antiplatelet agents (e.g., abciximab, clopidogrel, eptifibatide, ticlopidine, cilostazol, dipyridamole, sulfinpyrazone, tirofiban), beta blockers (e.g., acebutolol, betaxolol, carteolol, metoprolol, penbutolol, propranolol, atenolol, bisoprolol, esmolol, nadolol, pindolol, timolol), calcium channel blockers (e.g., amlopidine, felodipine, isradipine, nifedipine, verapamil, diltiazem, nicardipine, nimodipine, nisoldipine), diuretics, aldosterone blockers, loop diuretics (e.g., bumetanide, furosemide, ethacrynic acid, torsemide), potassium-sparing diuretics, thiazide diuretics (e.g., chlorothiazide, chlorthalidone, hydrochlorothiazide, hydroflumethiazide, methyclothiazide, metolazone, polythiazide, quinethazone, trichlormethiazide), inoptropics, bile acid sequestrants (e.g., cholestyramine, coletipol, colesevelam), fibrates (e.g., clofibrate, gemfibrozil, fenofibrate), statins (e.g., atorvastatinm, lovastatin, simvastatin, fluvastatin, pravastatin), selective cholesterol absorption inhibitors (e.g., ezetimibe), potassium channel blockers (e.g., amidarone, ibutilide, dofetilide), sodium channel blockers (e.g., disopyramide, mexiletine, procainamide, quinidine, flecainide, moricizine, propafenone), thrombolytic agents (e.g., alteplase, reteplase, tenecteplase, anistreplase, streptokinase, urokinase), vasoconstrictors, vasodilators (e.g., hydralazine, minoxidil, mecamylamine, isorbide dintrate, isorbide mononitrate, nitroglycerin).

In some embodiments, the nanoparticles associated with such compositions as described herein are further associated with (e.g., complexed, conjugated, encapsulated, absorbed, adsorbed, admixed) agents useful for determining the location of administered particles. Agents useful for this purpose include fluorescent tags, radionuclides and contrast agents.

Suitable imaging agents include, but are not limited to, fluorescent molecules such as those described by Molecular Probes (Handbook of fluorescent probes and research products), such as Rhodamine, fluorescein, Texas red, Acridine Orange, Alexa Fluor (various), Allophycocyanin, 7-aminoactinomycin D, BOBO-1, BODIPY (various), Calcien, Calcium Crimson, Calcium green, Calcium Orange, 6-carboxyrhodamine 6G, Cascade blue, Cascade yellow, DAPI, DiA, DID, Di1, DiO, DiR, ELF 97, Eosin, ER Tracker Blue-White, EthD-1, Ethidium bromide, Fluo-3, Fluo4, FM1-43, FM4-64, Fura-2, Fura Red, Hoechst 33258, Hoechst 33342, 7-hydroxy-4-methylcoumarin, Indo-1, JC-1, JC-9, JOE dye, Lissamine rhodamine B, Lucifer Yellow CH, LysoSensor Blue DND-167, LysoSensor Green, LysoSensor Yellow/Blu, Lysotracker Green FM, Magnesium Green, Marina Blue, Mitotracker Green FM, Mitotracker Orange CMTMRos, MitoTracker Red CMXRos, Monobromobimane, NBD amines, NeruoTrace 500/525 green, Nile red, Oregon Green, Pacific Blue. POP-1, Propidium iodide, Rhodamine 110, Rhodamine Red, R-Phycoerythrin, Resorfin, RH414, Rhod-2, Rhodamine Green, Rhodamine 123, ROX dye, Sodium Green, SYTO blue (various), SYTO green (Various), SYTO orange (various), SYTOX blue, SYTOX green, SYTOX orange, Tetramethylrhodamine B, TOT-1, TOT-3, X-rhod-1, YOYO-1, YOYO-3. In some embodiments, ceramides are provided as imaging agents. In some embodiments, SIP agonists are provided as imaging agents.

Additionally radionuclides can be used as imaging agents. Suitable radionuclides include, but are not limited to radioactive species of Fe(III), Fe(II), Cu(II), Mg(II), Ca(II), and Zn(II) Indium, Gallium and Technetium. Other suitable contrast agents include metal ions generally used for chelation in paramagnetic T1-type MIR contrast agents, and include di- and tri-valent cations such as copper, chromium, iron, gadolinium, manganese, erbium, europium, dysprosium and holmium. Metal ions that can be chelated and used for radionuclide imaging, include, but are not limited to metals such as gallium, germanium, cobalt, calcium, indium, iridium, rubidium, yttrium, ruthenium, yttrium, technetium, rhenium, platinum, thallium and samarium. Additionally metal ions known to be useful in neutron-capture radiation therapy include boron and other metals with large nuclear cross-sections. Also suitable are metal ions useful in ultrasound contrast, and X-ray contrast compositions.

Examples of other suitable contrast agents include gases or gas emitting compounds, which are radioopaque.

In some embodiments, the nanoparticles associated with such compositions as described herein are further associated with (e.g., complexed, conjugated, encapsulated, absorbed, adsorbed, admixed) a targeting agent. In some embodiments, targeting agents are used to assist in delivery of the nanoparticles associated with such compositions as described herein to desired body regions (e.g., bodily regions affected by a cardiovascular related disorder). Examples of targeting agents include, but are not limited to, an antibody, receptor ligand, hormone, vitamin, and antigen, however, the present invention is not limited by the nature of the targeting agent.

In some embodiments, the antibody is specific for a disease-specific antigen. In some embodiments, the receptor ligand includes, but is not limited to, a ligand for CFTR, EGFR, estrogen receptor, FGR2, folate receptor, IL-2 receptor, glycoprotein, and VEGFR. In some embodiments, the receptor ligand is folic acid.

In some embodiments, the nanoparticles associated with such compositions as described herein may be delivered to local sites in a patient by a medical device. Medical devices that are suitable for use in the present invention include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the agents are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (SCIMED LIFE SYSTEMS, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

In some embodiments, the present invention also provides kits comprising compositions as described herein. In some embodiments, the kits comprise one or more of the reagents and tools necessary to generate such compositions, and methods of using such compositions.

The compositions as described herein may be characterized for size and uniformity by any suitable analytical techniques. These include, but are not limited to, atomic force microscopy (AFM), electrospray-ionization mass spectroscopy, MALDI-TOF mass spectroscopy, ¹³C nuclear magentic resonance spectroscopy, high performance liquid chromatography (HPLC) size exclusion chromatography (SEC) (equipped with multi-angle laser light scattering, dual UV and refractive index detectors), capillary electrophoresis and get electrophoresis. These analytical methods assure the uniformity of the composition population and are important in the production quality control for eventual use in in vivo applications.

Where clinical applications are contemplated, in some embodiments of the present invention, the compositions are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight composition formulation may be administered using one or more of the routes described herein.

In preferred embodiments, the nanoparticles associated with such compositions as described herein are used in conjunction with appropriate salts and buffers to render delivery of the compositions in a stable manner to allow for uptake by target cells. Buffers also are employed when the compositions are introduced into a patient. Aqueous compositions comprise an effective amount of composition to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active compositions as described herein may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compositions as described herein in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, such compositions as described herein are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. The compositions also may be formulated as inhalants.

The present invention also includes methods involving co-administration of the compositions as described herein with one or more additional active agents. Indeed, it is a further aspect of this invention to provide methods for enhancing prior art therapies and/or pharmaceutical compositions by co-administering the compositions of this invention. In co-administration procedures, the agents may be administered concurrently or sequentially. In some embodiments, the compositions described herein are administered prior to the other active agent(s). The agent or agents to be co-administered depends on the type of condition being treated.

The present disclosure further provides kits comprising compositions comprising nanoparticles associated with such compositions as described herein or the ingredients necessary to synthesize the nanoparticles as described herein. In some embodiments, the kit includes all of the components necessary, sufficient or useful for administering such nanoparticles associated with such compositions as described herein.

### EXPERIMENTAL

The following examples are provided in order to demonstrate and further illustrate certain preferred embodiments and aspects of the present invention and are not to be construed as limiting the scope thereof. Use of the terms “I”, “our”, “we”, and similar terms, refers to the inventors.

### Example I

### Synthesis of CDN STING Agonist-Metal Coordination Polymeric Forms

c-di-AMP (CDA) were obtained from Invivogen and dissolved in water to obtain 2 mg/ml solution. ZnCl₂, MgCl₂, MnCl₂, CaCl₂), Al₂(SO₄)₃, CuCl₂, FeCl₂, FeCl₃, NiCl₂ and CoCl₂ (Sigma-Aldrich) was each dissolved in water to prepare 100 mM solution. To screen for metal ions that could coordinated with CDA, 5 or 10 equivalents (n/n) of metal ions were added to 2 mg/ml CDA solution. The mixture solution was incubated for 2 hours at room temperature. The resulting CDN-metal coordination polymeric forms could be observed under microscope. The degree of precipitation of CDN-metal solution could be measured by measuring absorbance at OD450 nm, OD500 nm, and OD 600 nm after substracting the background absorbance. Loading efficacy of CDN in the coordination structure were measured by quantifying the CDN in the supernatant and pellets after centrifugation 10000 xg, 15 min.

### Synthesis of CDN STING Agonist-Zn Co-Crystal Coordination Polymeric Forms

0.1, 0.5, 1, 2, 4, and 8 equivalents (n/n) of Zn²⁺ in 10 ul water were added to 20 ul 2 mg/ml CDA solution. The mixture solution was incubated for 2 hours at room temperature. The resulting CDN-metal coordination structures could be observed under microscope. After 4 hour, 80 ul of 5 mg/ml H33-PEG₂₀ₖ were added and resuspended. The mixture solution was incubated for another 96 hours and observed with microscopy.

### Synthesis of CDN STING Agonist-Zn/Mn Co-Crystal Coordination Polymeric Forms

0.1, 0.5, 1, 2, 4, and 8 equivalents (n/n) of Zn^{2+/}Mn²⁺(1:1) in 10 ul water were added to 20 ul 2 mg/ml CDA solution. The mixture solution was incubated for 2 hours at room temperature. The resulting CDN-metal coordination structure could be observed under microscope. After 4 hour, 80 ul of 5 mg/ml H33-PEG₂₀ₖ were added and resuspended. The mixture solution was incubated for another 96 hours and observed with microscope.

### Synthesis of CDN STING Agonist-Zn/Mn-H33-PEG Nanoscale Coordination Polymeric Forms

CDN STING agonist-Zn/Mn-H33-PEG nanoscale coordination polymeric forms was prepared by adding Zn^{2+/}Mn²⁺ to pre-mixed CDA and H33-PEG₂₀ₖ with stirring. Briefly, polyhistidine-PEG was added dropwise to CDA solution with continuous stirring. Then, 10-80 eq Zn^{2+/}Mn²⁺ were added to CDA/H33-PEG₂₀ₖ mixture under stirring and reacted for 12 hours. CDN STING agonist-Zn/Mn-H33-PEG nanoscale were isolated by centrifuge or centrifugal ultrafiltration (100kD). CDN STING agonist-Zn/Mn-H33-PEG nanoscale were further characterized by dynamic light scattering (DLS), transmission electron microscopy (TEM), and absorbance measurement.

### In Vitro Release Analysis

The release profiles of formulations were studied by a Slide-A-Lyzer™ MINI Dialysis Device, 20 KD MWCO (Thermo Scientific). Briefly, 0.5 ml formulation solution was filled in the cup with regenerated cellulose membrane and 14 ml release buffer (PBS) was put in the tube. After dialysis cup was inserted into the conical tube and capped, the device was incubated at 37° C. under continuous shaking (200 rpm). At the indicated time points, 300 ul of release media were collected and equal amount of fresh PBS was refilled. The concentration of CDN in the release medium was analyzed by HPLC (GPC). Finally, the release percentage was calculated based on the CDN concentration in the release buffer, volume of buffer, and the total CDN loading amount.

### Animal Studies

All animals were cared for following federal, state, and local guidelines. All work performed on animals was in accordance with and approved by the University Committee on Use and Care of Animals (UCUCA) at University of Michigan, Ann Arbor. Female Balb/c mice of age 6-8 weeks (Jackson Laboratories) were inoculated with 1.5×10⁵ CT26 colon cancer cells. When tumor size achieved ˜50 mm³, indicated drugs or formulations were administrated via the indicated route. Tumor size and survival were monitored every 2 or 3 days. Tumor size was calculated based on equation: volume=length× width²×0.5. Animals were euthanized when the tumor reached 1.5 cm in diameter or when animals became moribund with severe weight loss or un-healing ulceration.

### Results

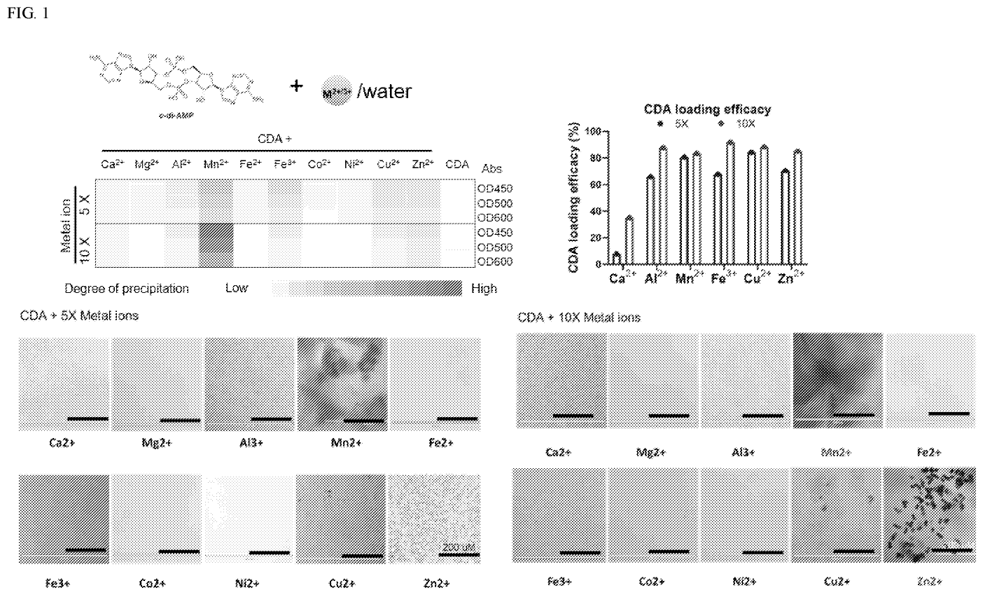

As shown in FIG. 1, after addition of metal ions into CDA solution, significant precipitation were observed for Mn²⁺, Al³⁺, Fe³⁺, Cu²⁺, Zn²⁺, Ca²⁺, but not for Mg²⁺, Fe²⁺, Co²⁺, Ni²⁺. The loading efficacy of CDA in the coordination structure is over 80% for Mn²⁺, Al³⁺, Fe³⁺,Cu²⁺, Zn²⁺. In the microscopy images, various coordination structures/polymorphs could be observed for Mn²⁺, Al³⁺, Fe³⁺,Cu²⁺, Zn²⁺, and Ca²⁺. Interestingly, Mn²⁺ formed the most significant coordination precipitation while Zn²⁺ formed defined crystal structures.

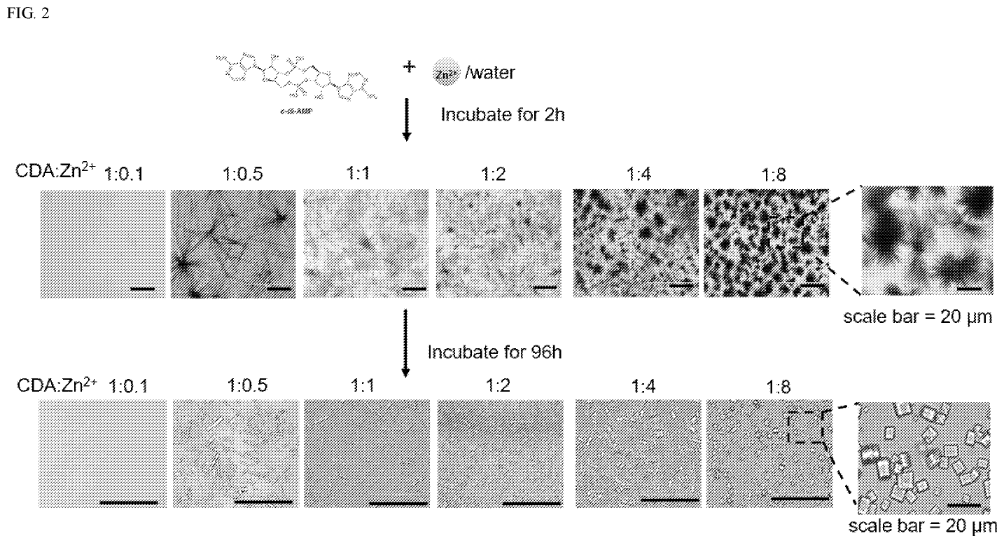

As shown in FIG. 2, when Zn²⁺ was added to CDA solution, needle-like crystals/polymorphs were observed when Zn/CDA was greater than 0.1:1 (n/n). On the other hand, 0.5 eq Zn²⁺ formed larger but less needle-like crystals, while 4 and 8 eq Zn²⁺ formed needle-like clusters. Interestingly, after incubation for another 96 h, we observed the formation of homogeneous cuboidal crystals/polymorphs. Adding varying amount of Zn²⁺ further tuned the length/width ratio of the crystals/polymorphs.

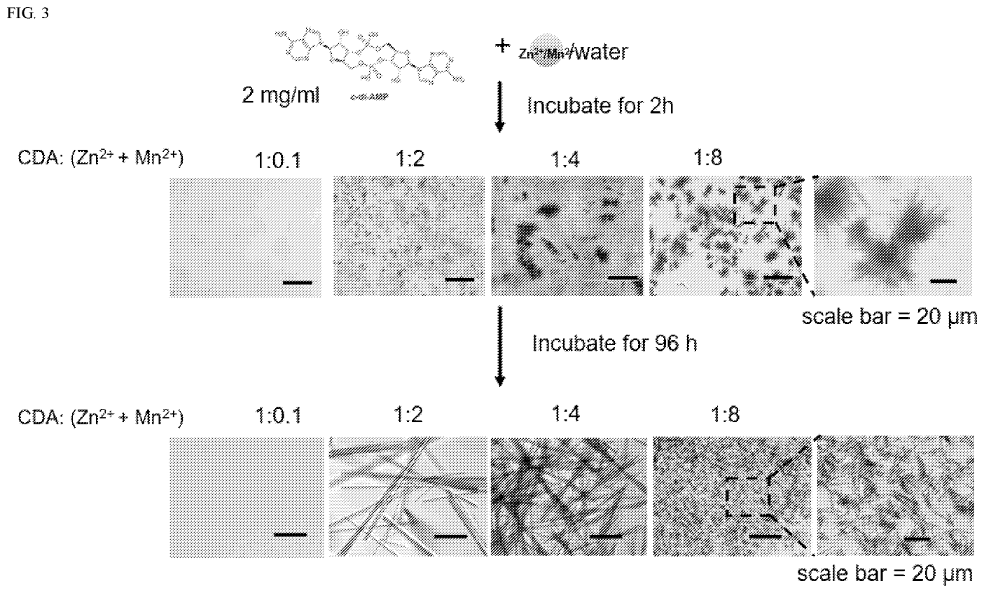

As shown in FIG. 3, when Zn²⁺+Mn²⁺ (1:1, n/n) were added to CDA solution, needle-like crystals/polymorphs were observed when (Zn²⁺+Mn²⁺)/CDA was greater than 0.1:1 (n/n). After incubation for another 96 h, we observed that Zn²⁺+Mn²⁺ formed homogeneous needle-like crystal with CDA.

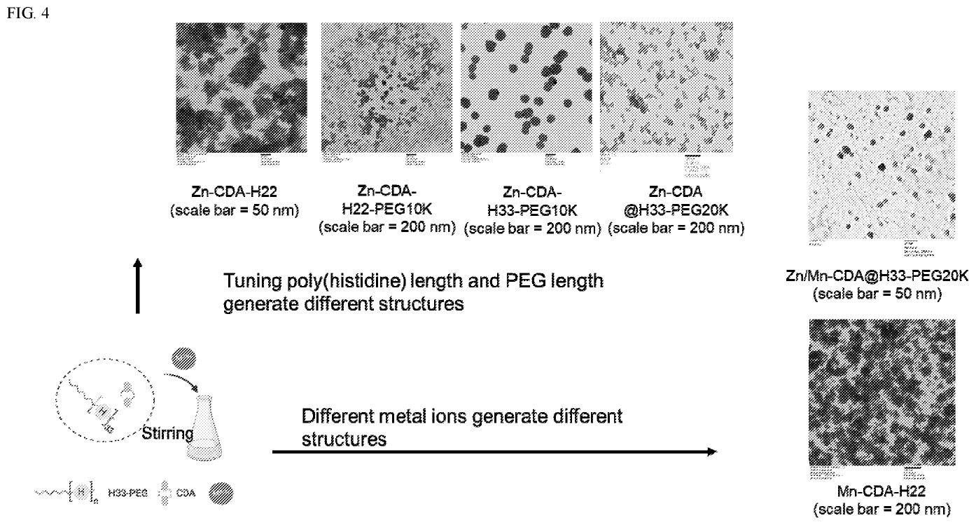

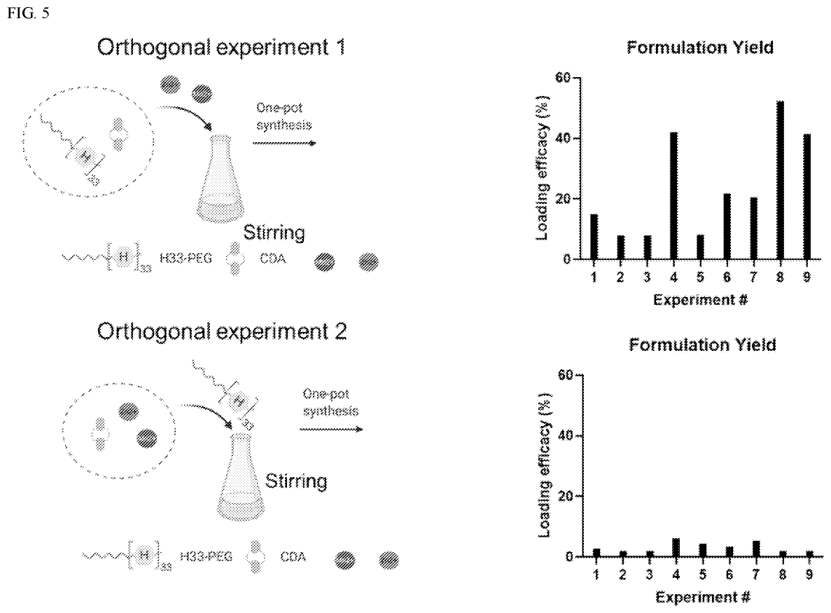

In contrast to the large coordination structures shown in FIG. 1-3, when CDA, H33-PEG₂₀ₖ, and Zn^{2+/}Mn²⁺ were mixed in order under continuous stirring, we observed the formation of nanostructures (FIG. 4). Interestingly, by changing the length of poly(histidine) and PEG in polyhistidine-PEG copolymer and metal ions types, distinct nano coordination structures were formed. In particular, H33-PEG₂₀ₖ and Zn or/and Mn generated homogeneous spherical coordination nanostructures.

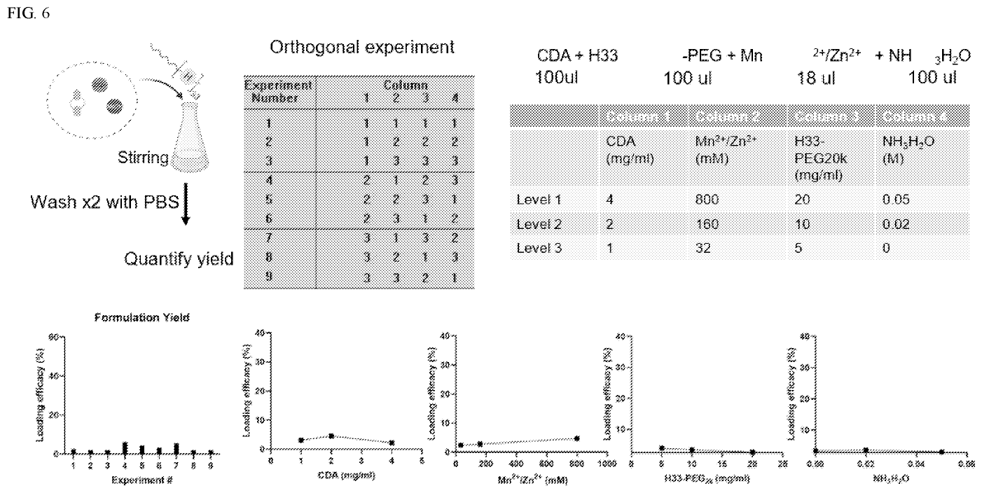

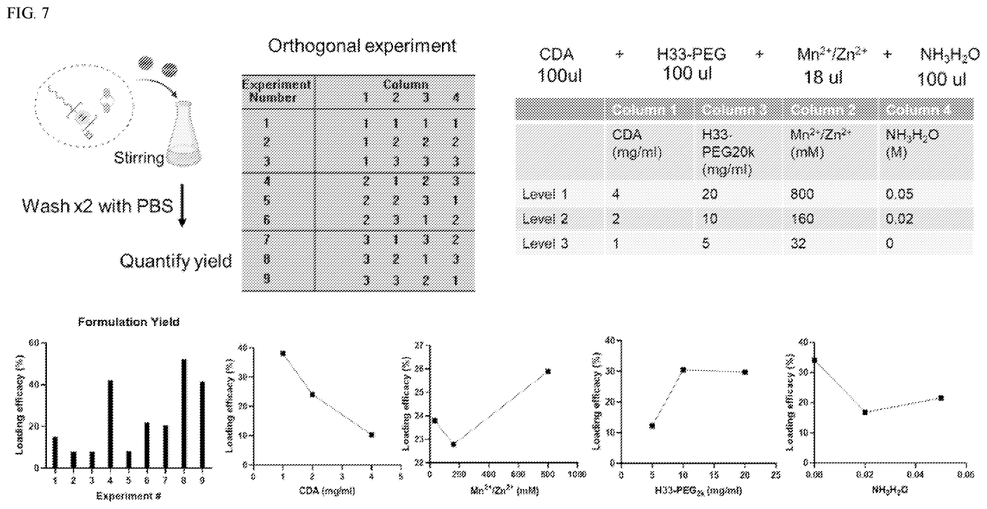

To optimize the condition for formulating stable CDN STING agonist-Zn/Mn-H33-PEG nanoparticle (CZMP), we conducted 2 sets of orthogonal experiments (FIG. 5-7). In the orthogonal experiment 1, varying concentrations of H33-PEG were added to CDA/M²⁺ mixture in various ratios and washed with PBS. In the orthogonal experiment 2, varying concentrations of M²⁺ were added to CDA/H33-PEG20k mixture in various ratios and washed with PBS. Surprisingly, adding H33-PEG to CDA/M²⁺ mixture resulted in low yields (FIG. 6). In contrast, adding M²⁺ to CDA/H33-PEG₂₀ₖ mixture efficiently formed stable formulations with high yields (FIG. 7). In addition, we have determined other crucial factors for forming nanostructures with high yields: 1) the concentration of CDN STING agonist in the synthesis should be more than 0.001 mg/ml. 2) The concentration of H33-PEG₂₀ₖ should be more than 0.01 mg/ml for effective formulation. 3) The molar ratio of metal ion/CDN should be more than 0.01.

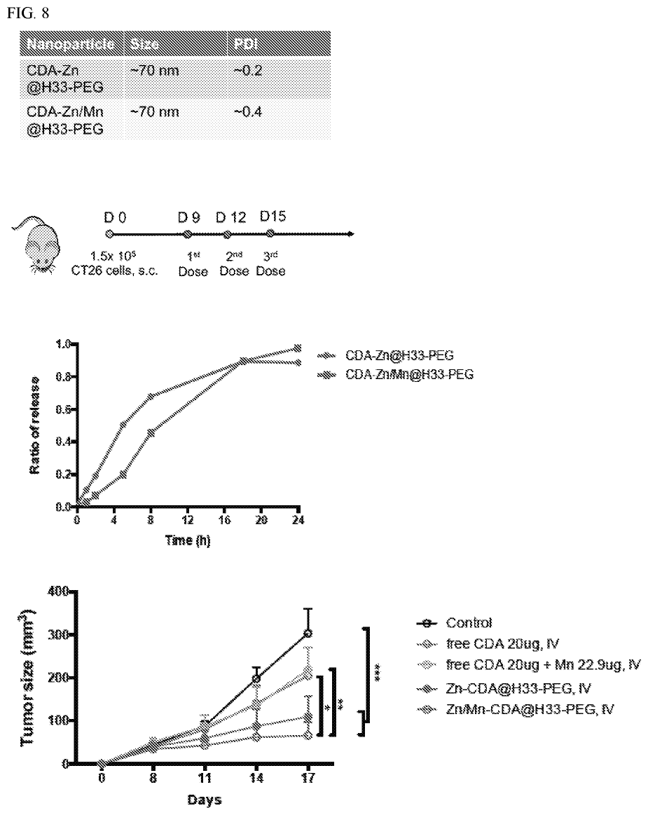

To evaluate the activity of CDN STING agonist-Zn/Mn-H33-PEG nanoscale coordination polymeric forms, we established a CT26 subcutaneous tumor model and administrated CDA-Zn/Mn-H33-PEG intravenously (IV) at day 9, 12, 15 after tumor inoculation. As shown in FIG. 8, dynamic light scattering analyses indicated that CDA-Zn-H33-PEG and CDA-Zn/Mn-H33-PEG have average hydrodynamic sizes of˜70 nm. We observed steady drug release from CDA-Zn-H33-PEG and CDA-Zn/Mn-H33-PEG. After 3 dose of systemic administration, CDA-Zn/Mn-H33-PEG showed significantly improved antitumor effects, compared with equal dose of CDA, free CDA+Mn²⁺, or untreated control. Similarly, CDA-Zn-H33-PEG also demonstrated antitumor effects. These results show that the coordination polymeric forms can significantly improve the activity and therapeutic effects of CDN STING agonists.

### Example II

This example describes vaccination studies with CDN STING agonist-metal coordination polymeric forms.

Experiments were conducted that examined CDN STING agonist-metal coordination polymeric forms for vaccine applications. We prepared CDN STING agonist-metal coordination polymeric forms and admixed with SARS-CoV-2 viral antigens and performed immunogenicity studies in mice.

### Animal Vaccination Schedule

CDN STING agonist-metal coordination polymeric forms (termed CDA-Mn/Zn@H33-PEG) were prepared as shown in Example I. Mice (BALB/c) were vaccinated with CDA-Mn/Zn@H33-PEG or CDA, each admixed with S1 or RBD protein from SARS-CoV-2. Vaccinations were performed three times at subcutaneous tail base with 2 weeks intervals. 2 weeks after each vaccination and 4 and 8 weeks after the last vaccination, sera were collected for antibody titer measurement by ELISA. In a separate study, spleens were harvested 1 week after the last vaccination for ELISPOT assay.

### ELISA

96-well plates were pretreated overnight with 0.1 μg of RBD (dissolved in carbonate-bicarbonate buffer) per well. After removing RBD, wells were washed three times with wash solution and further incubated with blocking solution for 2 hours in RT, then washed three times with wash solution. Next, sera samples were put into the plates. Sera samples were serially diluted by 1:4 or 1:5 six times with a starting dilution of 1:100. After incubating the samples for 1 hour in RT, samples were removed, followed by three times of washing. Next, 1:1000-diluted HRP-conjugated anti-mouse IgG antibodies were added and incubated for 1 hour in RT. After the incubation, antibodies were removed and the wells are washed 4 times with wash solution, then substrate solution was added and incubated for 10 min or until the reacting solution colors were developed in RT. The reaction was stopped by adding 1 M H2SO4, then the plates were read at a wavelength of 450 nm.

### ELISPOT

A day before spleen harvest, a 96-well ELISPOT plates were coated with capture antibody (anti-IFN-γ antibody). After overnight incubation in 4C, wells were washed with blocking solution, followed by incubation with blocking solution for 2 hours. Next the blocking solution was removed, and RBM peptides (Table 1) were added to the wells in 10 μg/ml concentration.

TABLE 1
RBD peptide sequences used in the ELISPOT assays

| Sequence | AA seq. no. |
|---|---|
| TVCGPKKSTNLVKNKCVNF (SEQ ID NO: 20) | 523-541 |
| LSFELLHA (SEQ ID NO: 21) | 513-520 |
| TNGVGYQPYR (SEQ ID NO: 22) | 500-509 |
| CNGVEGFNCYFPLQSYG (SEQ ID NO: 23) | 480-496 |
| NYLYRLFRKSNLKPFERDISTEIY (SEQ ID NO: 24) | 450-473 |
| SNNLDSKVGGNYNYLYRLFRK (SEQ ID NO: 25) | 438-458 |
| VYADSFVIRGDEVRQIAPGQTGKIADYNYKLPDDFT (SEQ ID | 395-430 |
| NO: 26) | |
| SASFSTFKCYGVSPTKLNDLCFTN (SEQ ID NO: 27) | 371-394 |

Spleens were harvested 1 week after the last vaccination. Each spleen was placed on top of a 40 μm strainer then mashed with a plunger. The strainer was washed with PBS to let splenocytes pass through into a collection tube. After centrifugation at 300 g for 5 min, each cell pallet was resuspended with ACK lysis buffer and incubated for 5 min in RT. Next, the cells were centrifuged and washed with PBS twice. Resulting cells were counted and 2×10⁵ cells were seeded in each well of the 96-well ELISPOT plate containing the RBM peptide and incubated in 37C for overnight.

Next, cell suspensions were aspirated out and the wells were washed twice with deionized water, followed by three times of washing with wash solution. Detection antibody was added and incubated for 2 hours in RT. After incubation, antibodies were removed, wells were washed three times with wash buffer, then HRP-conjugated streptavidin was added and incubated for 1 hour in RT. Following incubation, well were washed four times with wash buffer and two times with PBS. Substrate solution was added and incubated until spots are developed in the wells. The wells were washed with water to stop the development. After air-drying overnight in RT, the plates were imaged with an ELISPOT reader.

### Result

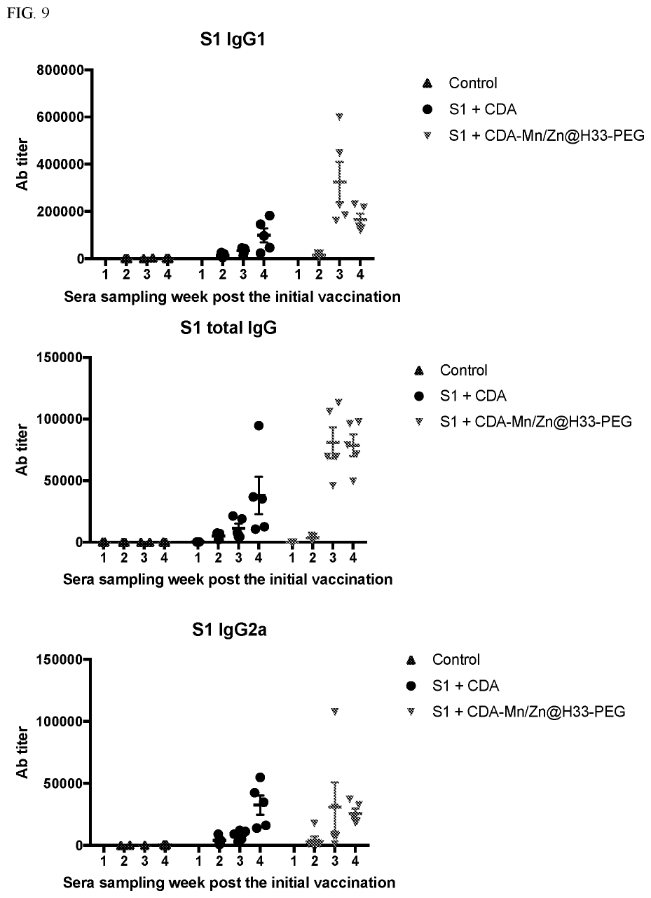

CDA-Mn/Zn@H33-PEG admixed with S1 protein from SARS-CoV-2 generated robust S1-specific antibody responses (FIG. 9). We detected significant elevated levels of S1-specific serum IgG, IgG1, and IgG2a titers in mice immunized with S1+CDA-Mn/Zn@H33-PEG, compared with those immunized with free mixture of S1+CDA.

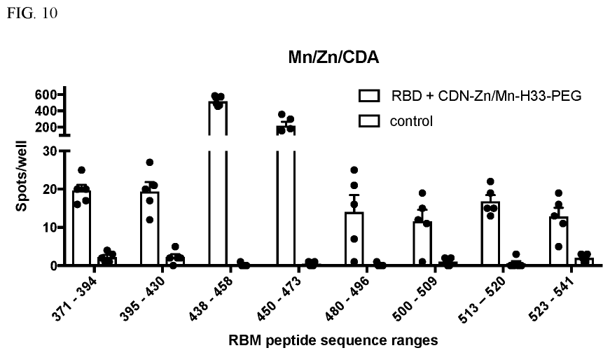

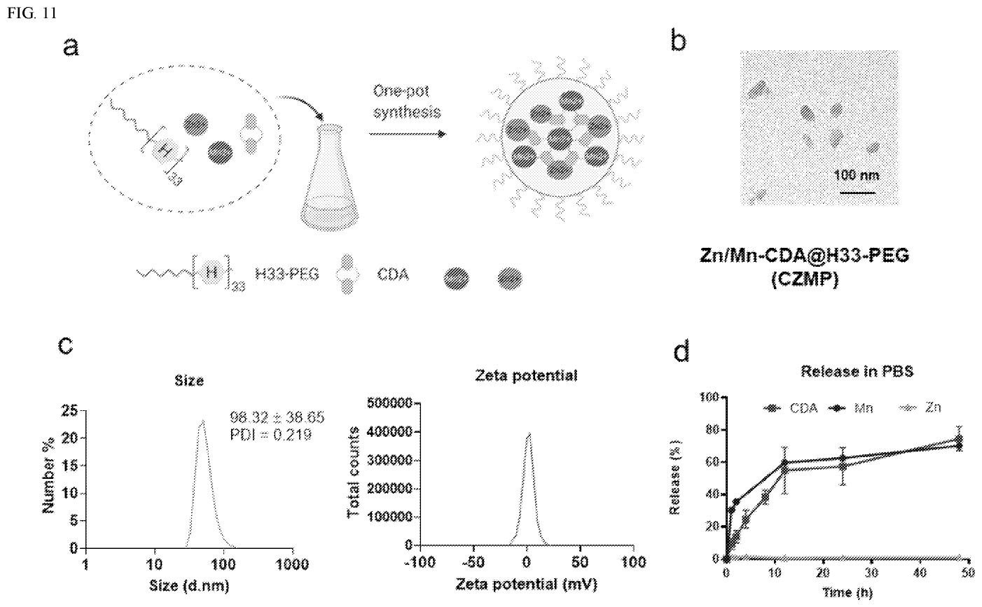

In a subsequent study, CDA-Mn/Zn@H33-PEG admixed with RBD protein from SARS-CoV-2 were used to vaccinated BABL/c mice on days 0, 14, and 28. ELISPOT assay was performed on day 35 to examine RBD-sequence specific T-cell responses among splenocytes. CDA-Mn/Zn@H33-PEG admixed with RBD generated robust RBD-specific IFN-gamma+ T-cell responses in BABL/c mice (FIG. 10). In particular, RBD sequences 438-458 and 450-473 were immunodominant epitopes in BABL/c mice.

### Example III

In order to design a nanoparticle delivery system of STING agonist, we also used CZMP for local and systemic delivery of CDN STING agonist since Mn has a potentiating effect on STING agonist and H33-PEG is biocompatible and ionizable for endosome escape. Using optimized condition, by one-pot synthesis, we obtained homogeneous CZMP nanoparticles with a DLS size of 98.3±38.7 nm and near neutral surface charge (FIG. 11a-c). CZMP nanoparticles exhibited steady release of CDA and Mn²⁺, but minimal release of Zn²⁺ (FIG. 11d).

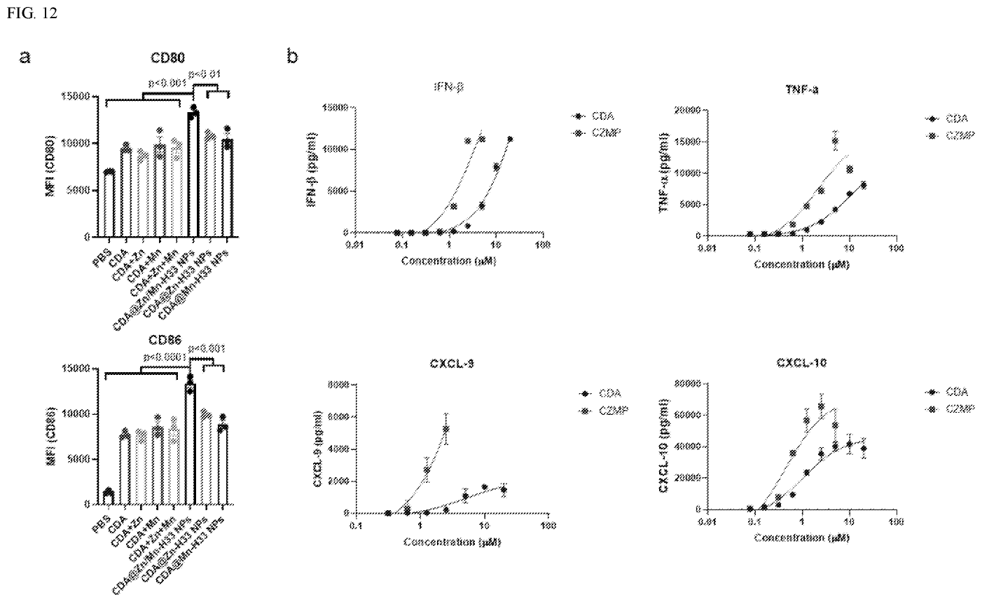

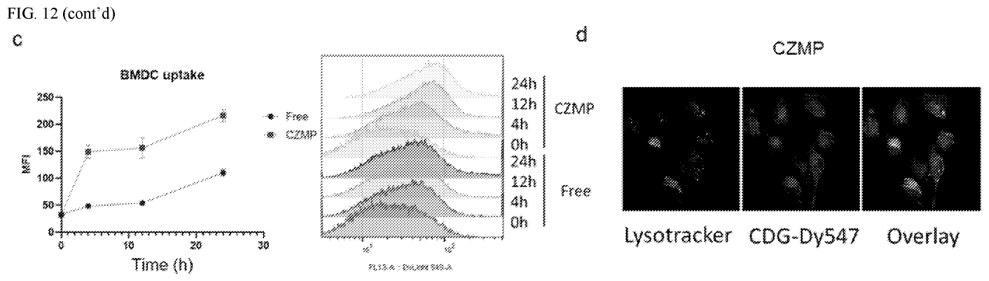

We further tested the bioactivity of CAMP in vitro (FIG. 12). Compared with CDA, CDA+Mn²⁺ and/or Zn²⁺, Zn-CDA@H33-PEG, Mn-CDA@H33-PEG BMDC, CZMP significantly elevated the expression levels of CD80 and CD86 on BMDCs, which indicated more potent STING activation (FIG. 12a). In THP1-STING-R232, we also found CZMP induced higher IFN-I production and other related cytokines in a much low concentration of STING agonist (FIG. 12b). Strong STING activation can be explained by the increased cellular uptake of STING agonists by BMDCs after CZMP treatment (FIG. 12c) and efficient endosome escape (FIG. 12d).

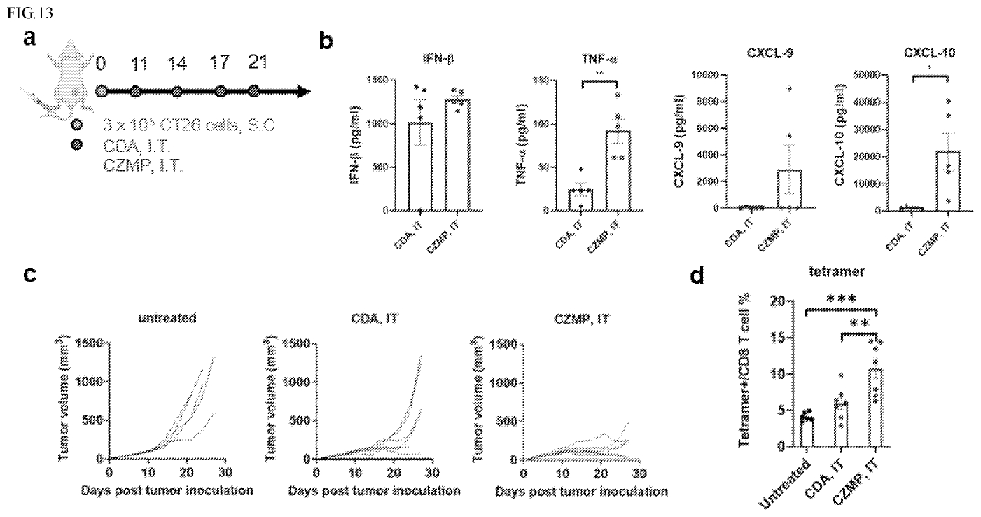

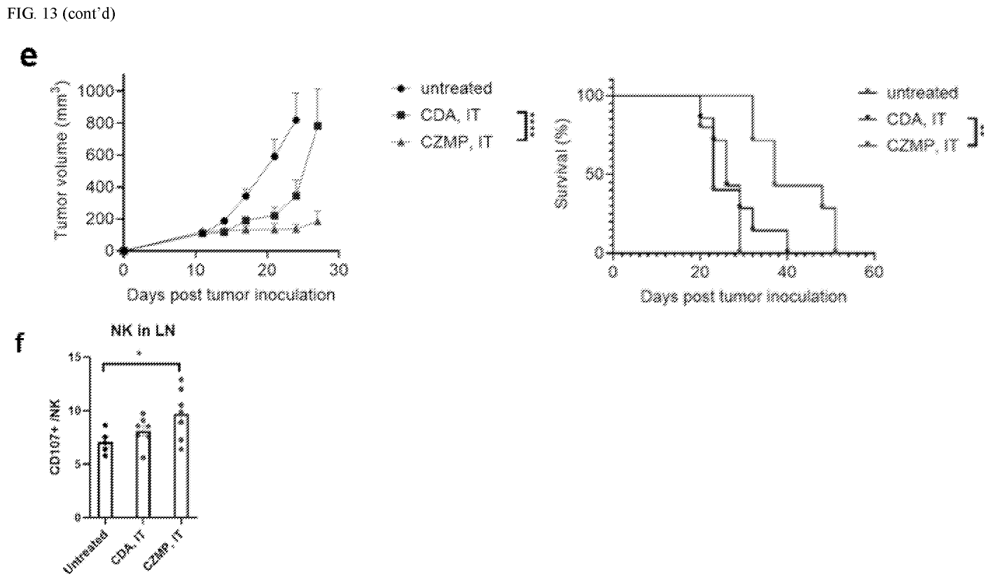

To evaluate the therapeutic potential of CZMP in vivo after local administration, CT26 tumor-bearing BABL/c mice were treated with I.T. administration of CZMP or free CDA on days 11, 14, 17, and 21. Compared with the free CDA, CZMP increased the serum levels of IFN-β, TNF-α, CXCL-9, and CXCL-10 (FIG. 13b), demonstrating higher immune activation. Importantly, CZMP administered I.T. significantly inhibited CT26 tumor growth and prolonged the survival of mice (FIGS. 13c, e). Flow cytometry analysis performed on day 27 showed that CZMP I.T. therapy significantly expanded NK cell population in PBMCs and induced high NK cell activation in tumor-draining lymph node (FIG. 13d, f). Similar therapeutic response of CZMP was also observed in B16F10 melanoma-bearing mice. CZMP I.T. therapy efficiently inhibited B16F10 tumor growth with significantly enhanced therapeutic efficacy, compared with free CDA (FIGS. 13g-i). Altogether, these results suggest that CZMP is a simple yet efficient formulation for systemic delivery of STING agonist for cancer immunotherapy.

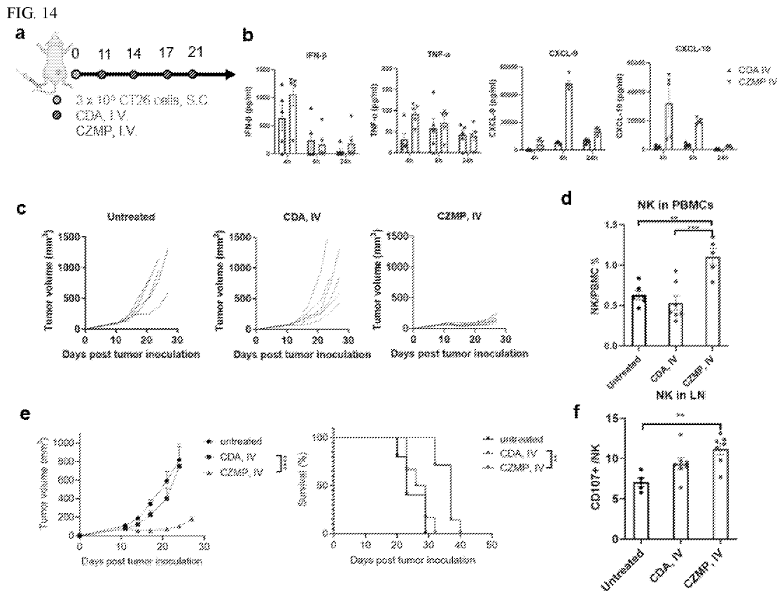

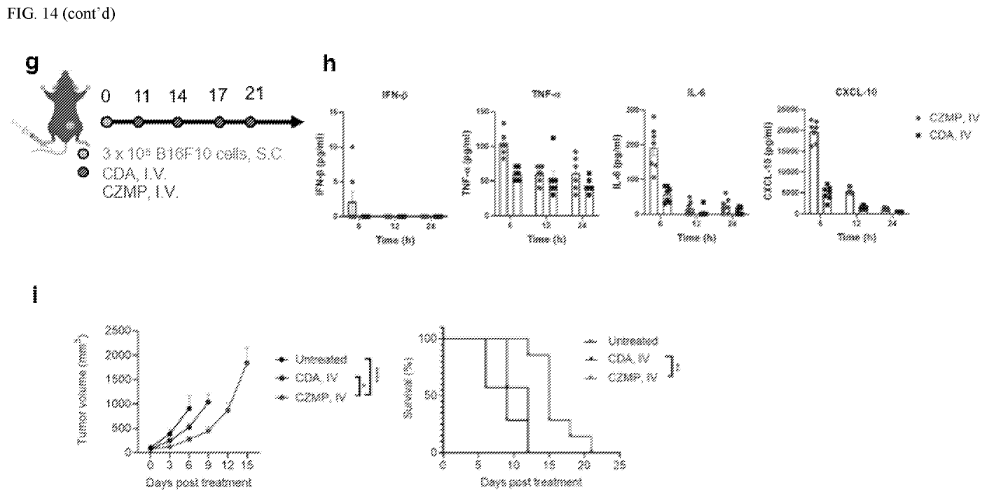

To evaluate the therapeutic potential of CZMP in vivo after systemic administration, CT26 tumor-bearing BABL/c mice were treated with I.V. administration of CZMP or free CDA on days 11, 14, 17, and 21. Compared with the free CDA, CZMP increased the serum levels of IFN-β, TNF-α, CXCL-9, and CXCL-10 (FIG. 14b) demonstrating higher immune activation. Importantly, CZMP administered I.V. significantly inhibited CT26 tumor growth and prolonged the survival of mice (FIG. 14c, e). Flow cytometry analysis performed on day 27 showed that CZMP I.V. therapy significantly expanded NK cell population in PBMCs and induced high NK cell activation in tumor-draining lymph nodes (FIGS. 14d, f). Similar therapeutic response of CZMP was also observed in B16F10 melanoma-bearing mice. CZMP I.V. therapy efficiently inhibited B16F10 tumor growth with significantly enhanced therapeutic efficacy, compared with free CDA (FIGS. 14g-i). Altogether, these results show that CZMP is a simple yet efficient formulation for systemic delivery of STING agonist for cancer immunotherapy.

### Example IV

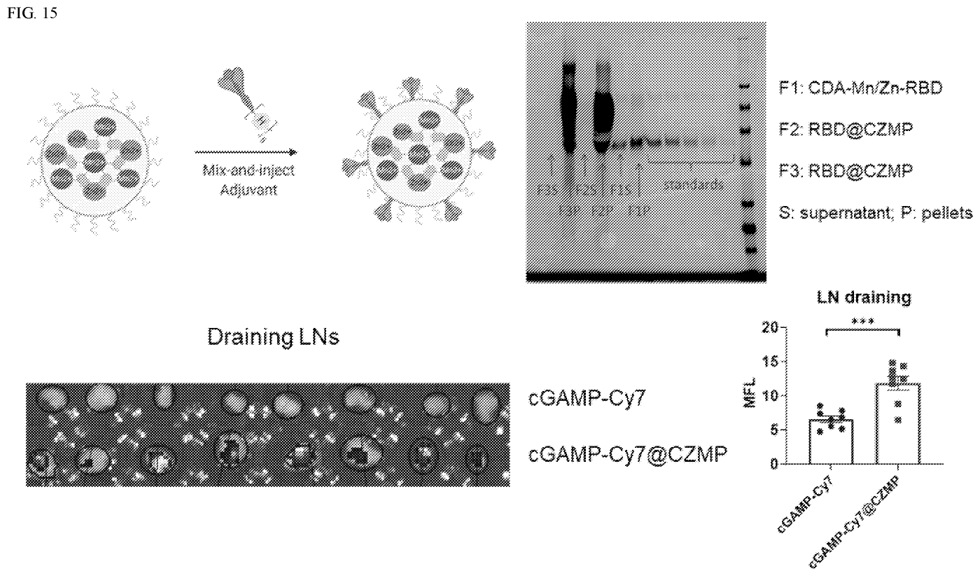

STING agonists have been reported as efficient vaccine adjuvants for vaccines of cancer and infectious diseases. Therefore, we further tested whether CZMP could be serve as effective vaccine adjuvant for COVID-19 vaccine. Interestingly, polyhistidine-tag technology are widely used for protein isolation and a large portion of recombinant proteins are grafted with polyhistidine-tags. Using the sample histidine-Zn/Mn coordination interaction, we can load various antigen with polyhis-tag onto CZMP. To demonstrate this, we added COVID-19 S1 protein RBD with 6×his tag into CZMP and quantified the protein loading after pulling down the nanoparticles. As shown in FIG. 12, >90% RBD could be effectively loaded onto CZMP based on SDS-PAGE result. As shown in the IVIS imaging result, we also found CZMP after subcutaneous injection drained to lymph node effectively (FIG. 15). This indicates CZMP can home to lymph nodes for effective delivery of antigens to B cell and antigen-presenting cells.

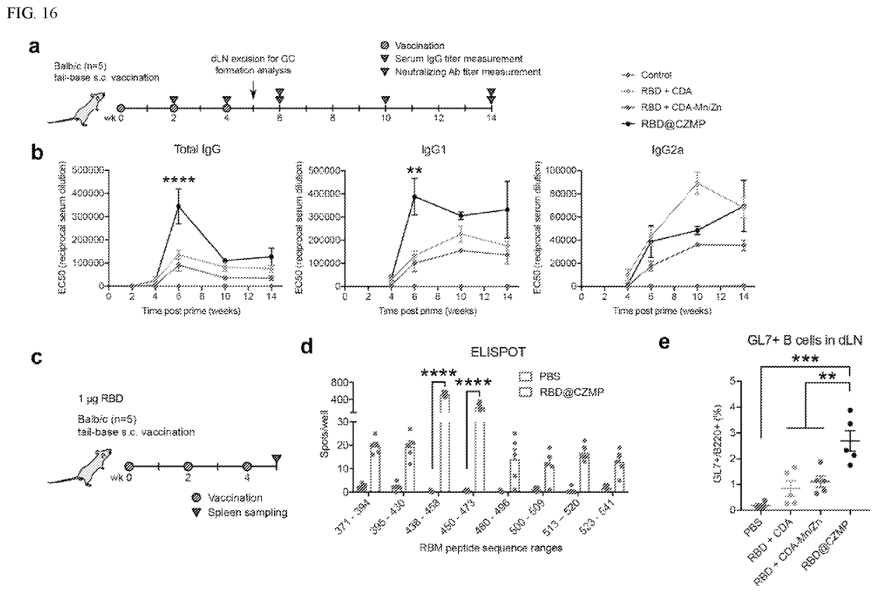

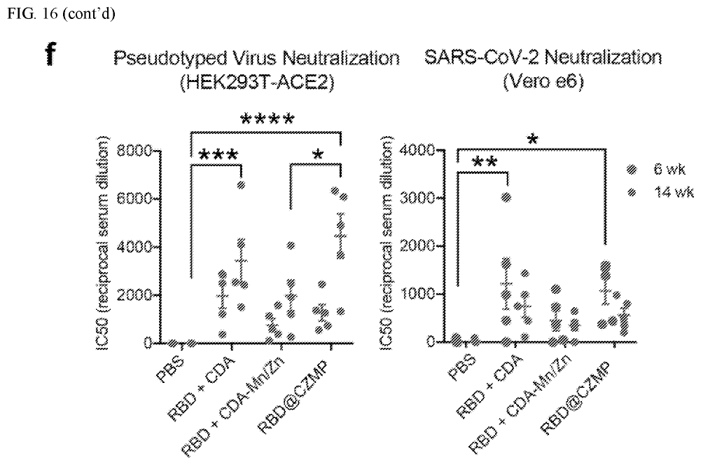

To evaluate the vaccine response of RBD@CZMP in vivo, Balb/c mice were vaccinated three times at tail base with 2 week intervals. 2 weeks after each vaccination and 4 and 8 weeks after the last vaccination, sera samples were collected for antibody titer measurement by ELISA (FIG. 16a). As shown in FIG. 16b, CZMP significantly increased the neutralizing antibody production. In a separate study, spleens were harvested 1 week after the last vaccination for ELISPOT assay (FIG. 16c-b). We tested T cell response induced by RBD@CZMP to a panel of epitopes within RBD. Strikingly, we found very high level of T cell response was induced (as high as 400/0.2M splenocytes), especially against sequence 438-458, 450-473. The ELISPOT study was also repeated in another separate study in comparison with RBD, RBD+free CDA, and RBD+free CDA+Mn+Zn. RBD@CZMP induced significantly higher T cell response. In the same study, we also observed improved germinal center formation after RBD@CZMP vaccination (FIG. 16e). Finally, we have also tested whether the antibody response induced by RBD@CZMP could neutralize SARS-CoV-2 virus infection. As shown in FIG. 16f, we treated the immunized mouse sera of week 6 and 14 to reporter cells and added SARS-CoV-2 pseudovirus or SARS-CoV-2 virus. We found RBD@CZMP induced effective neutralizing antibody to neutralize SARS-CoV-2 pseudovirus and SARS-CoV-2 virus. These data indicate CZMP may serve as a potent vaccine adjuvant platform for COVID-19 vaccine and other vaccines.

Lastly, we also evaluated the safety profile of CZMP platform. After either IV or IT injection, we have not observed any apparent body weight loss, abnormal serum chemistry, and histological changes. Overall, CZMP is safe and effective formulation of STING agonists for cancer immunotherapy and vaccine adjuvant.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A composition comprising crystalline polymorphic forms of one or more STING agonists, wherein each of the crystalline polymorphic forms of the one or more STING agonists comprises:
one or more STING agonists mixed with one or more metal ions selected from Zn²⁺, Mn²⁺, Al³⁺, Fe³⁺, and Cu²⁺,
wherein each of the one or more STING agonists mixed with the one or more metal ions is associated with a poly(histidine)-glycol moiety.

2. The composition of claim 1,
wherein the poly(histidine) is poly(histidine₃₃) (H33) and the glycol is polyethylene glycol (PEG) resulting in a H33-PEG moiety,
wherein the PEG length is between 1-500 repeat units,
wherein the polyhistidine₃₃ length is between 1-50 repeat units.

3. (canceled)

4. (canceled)

5. The composition of claim 1,
wherein the concentration of the one or more STING agonists within the composition is between 0.01 and 5 mg/ml,
wherein the molar ratio of one or more metal ions to the one or more STING agonists is more than 0.1.

6. The composition of claim 2, wherein the concentration of H33-PEG is equal to or more than 1.3 mg/ml within each of the crystalline polymorphic forms of the one or more STING agonists.

7. The composition of claim 1,
wherein the one or more STING agonists are cyclic dinucleotides, wherein the cyclic dinucleotides are independently selected from cGAMP, cdiAMP, cdiGMP, and cAIMP, or
wherein the one or more STING agonists are independently selected from 5,6-Dimethylxanthenone-4-acetic acid (DMXAA), methoxyvone, 6,4′-dimethoxyflavone, 4′-methoxyflavone, 3′,6′-dihydroxyflavone, 7,2′-dihydroxyflavone, daidzein, formononetin, and retusin 7-methyl ether, or any derivatives thereof, or
wherein the one or more STING agonists are independently selected from 2′3′-cGAMP, 3′3′-cGAMP, c-di-AMP, c-di-GMP, cAIMP, cAIMP Difluor, cAIM(PS)2, Difluor (Rp/Sp), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, SB11285, STING-agonist-C11, STING agonist-1, STING agonist G10, and Gemcitabine, or
wherein the one or more STING agonists are independently selected from 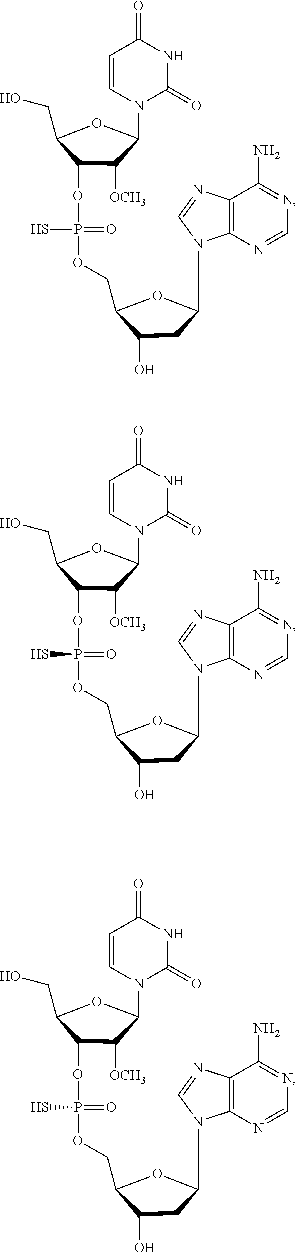 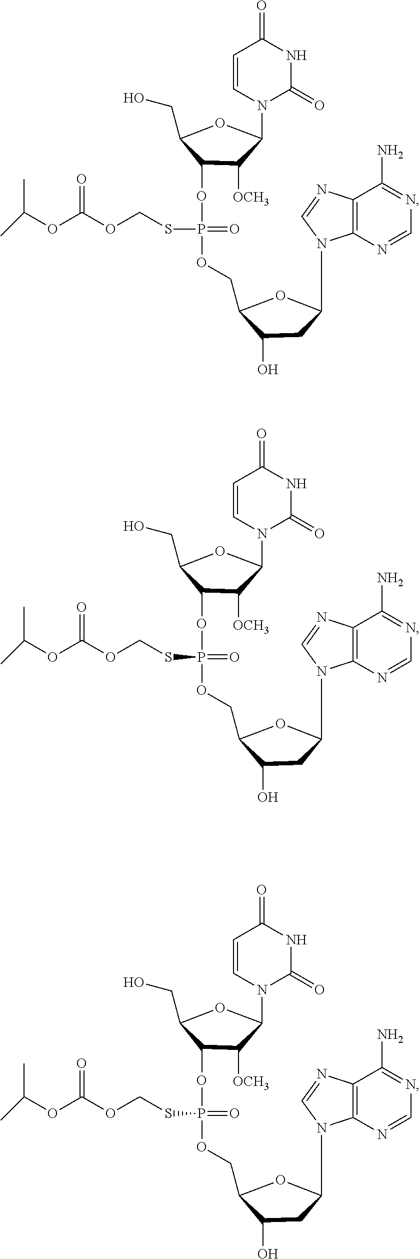
SB11285 (Spring Bank Pharmaceuticals), Gemcitabine 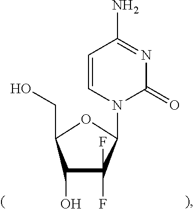
STING-agonist-C11 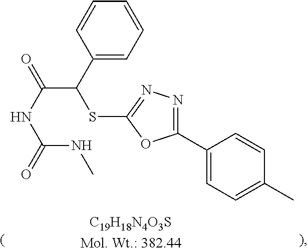 [SMILES: CNC(=O)NC(=O)C(Sc1nnc(-c2ccc(C)cc2)o1)c1ccccc1 ; "C19H18N4O3S" ; "Mol. Wt.: 382.44"]
STING agonist-1 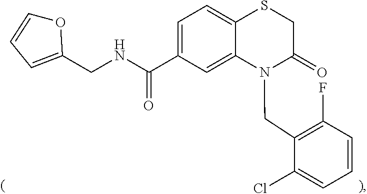 [SMILES: O=C(NCc1ccco1)c1ccc2c(c1)N(Cc1c(F)cccc1Cl)C(=O)CS2]
STING agonist G10 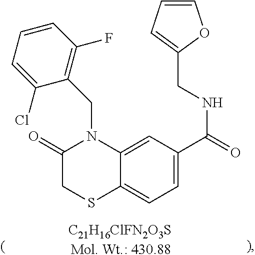
2′3′-cGAMP, 3′3′-cGAMP, c-di-AMP, c-di-GMP, cAIMP, cAIMP Difluor, cAIM(PS)2, Difluor (Rp/Sp), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, cGAMP, 2′3′-cGAMP, 2′2′-cGAMP, 3′3′-cGAMP, cGAM(PS)2, 2′3′-cGAM(PS)2(Rp/Sp), 2′2′-cGAM(PS)2, 2′3′-cGAM(PS)2, cGAMP Fluorinated, 3′3′-cGAMP Fluorinated, 2′3′-cGAMP Fluorinated, 2′2′-cGAMP Fluorinated, c-di-AMP, 2′3′-cdAMP, 2′2′-cdAMP, 3′3′-cdAMP, c-di-AM(PS)2, 2′3′-c-di-AM(PS)2 (Rp,Rp), 2′2′-c-di-AM(PS)2, 3′3′-c-di-AM(PS)2, c-di-AMP Fluorinated, 2′3′-cdAMP Fluorinated, 2′2′-cdAMP Fluorinated, 3′3′-cdAMP Fluorinated, cdGMP, 2′3′-cdGMP, 2′2′-cdGMP, 3′3′-cdGMP, c-di-GM(PS)2, 2′3′-c-di-GM(PS)2, 2′2′-c-di-GM(PS)2, 3′3′-c-di-GM(PS)2, cdGMP Fluorinated, 2′3′-cdGMP Fluorinated, 2′2′-cdGMP Fluorinated, 3′3′-cdGMP Fluorinated, cAIMP, 2′3′-cAIMP, 2′2′-cAIMP, 3′3′-cAIMP, cAIMP Difluor (3′3′-cAIMP Fluorinated, 2′3′-cAIMP Fluorinated, 2′2′-cAIMP Fluorinated, cAIM(PS)2 Difluor, 3′3′-cAIM(PS)2 Difluor (Rp/Sp), 2′3′-cAIM(PS)2 Difluor, 2′2′-cAIM(PS)2 Difluor, c-di-IMP, 2′3′-cdIMP, 2′2′-cdIMP, 3′3′-cdIMP, c-di-IM(PS)2, 2′3′-c-di-IM(PS)2, 2′2′-c-di-IM(PS)2, 3′3′-c-di-IM(PS)2, c-di-IMP Fluorinated, 2′3′-cdIMP Fluorinated, 2′2′-cdIMP Fluorinated, and 3′3′-cdIMP Fluorinated, amidobenzimidazole (ABZI)-based compounds, SR-717-based compounds, and MSA-2-based compounds.

8-10. (canceled)

11. The composition of claim 1, wherein the composition is generated via a one-pot reaction.

12. A method, comprising:
administering to a subject a therapeutically effective amount of the composition of claim 1,
wherein the method is for one or more of:
stimulating an innate immune response in the subject,
treating, preventing and/or ameliorating the symptoms of cancer in the subject,
treating, preventing, prophylactically vaccinating, and/or ameliorating the symptoms of infection in the subject, and
treating, ameliorating and/or preventing acute respiratory distress syndrome and/or pneumonia in the subject.

13. The method of claim 12, wherein the administration results in an innate cytokine response mediated through cytokines in the subject, wherein the innate cytokine response is mediated through type 1 interferon.

14-16. (canceled)

17. The method of claim 12, wherein an additional agent is co-administered with the composition, wherein the additional agent is selected from the group consisting of an adjuvant, a chemotherapeutic agent, an anti-immunosuppressive agent, an immunostimulatory agent, and an antigen.

18. The method of claim 17,
wherein the immunostimulatory agent is selected from anti-CTLA-4 antibody, anti-PD-1, anti-PD-LI, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, and inhibitors of IDO:
wherein the chemotherapeutic agent is selected from aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (TAXOL), pilocarpine, prochloroperazine, rituximab, tamoxifen, taxol, topotecan hydrochloride, trastuzumab, vinblastine, vincristine and vinorelbine tartrate.

19-23. (canceled)

24. The method of claim 12, wherein the composition is further associated with with one or more agents configured to target cancer cells.

25. The method of claim 24, wherein the agent configured to target cancer cells is a tumor antigen selected from the group consisting of alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pm1-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGS), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), human EGFR protein or its fragments, such as human EGFR residues 306-325 (SCVRACGADSYEMEEDGVRK (SEQ ID NO: 12)) and residues 897-915 (VWSYGVTVWELMTFGSKPY (SEQ ID NO: 13)), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, TPS, WT1 (and WT1-derivaed peptide sequences: WT1 126-134 (RMFP NAPYL (SEQ ID NO: 14)), WT1 122-140 (SGQARMFPNAPYLPSCLES (SEQ ID NO: 15)), and WT1 122-144 (SGQARMFPNAPYLPSCLESQPTI (SEQ ID NO: 16)), MUC1 (and MUC1-derived peptides and glycopeptides such as RPAPGS (SEQ ID NO: 17), PPAHGVT (SEQ ID NO: 18), and PDTRP (SEQ ID NO: 19)), LMP2, EGFRvIII, Idiotype, GD2, Ras mutant, p53 mutant, Proteinase3 (PRI), Survivin, hTERT, Sarcoma translocation breakpoints, EphA2, EphA4, LMW-PTP, PAP, ML-IAP, AFP, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, sLe(animal), CYP1B1, PLAC1, GM3, BORIS, Tn, GloboH, NY-BR-1, RGS5, SART3, STn, Carbonic anhydrase IX, PAX5, OY-TES1, Sperm protein 17, LCK, HMWMAA, AKAP-4, XAGE 1, B7H3, Legumain, Tie 2, Page4, VEGFR2, MAD-CT-1, FAP, PDGFR-alpha, PDGFR-β, MAD-CT-2, Fos-related antigen 1, ERBB2, Folate receptor 1 (FOLR1 or FBP), IDH1, IDO, LY6K, fins-related tyro-sine kinase 1 (FLT1, best known as VEGFR1), KDR, PADRE, TA-CIN (recombinant HPV16 L2E7E6), SOX2, aldehyde dehydrogenase, and any derivative thereof.

26. (canceled)

27. The method of claim 12,
wherein the cancer is one or more selected from bladder cancer, brain cancer, breast cancer, cervical cancer, ovarian cancer, colo-rectal cancer, esophageal cancer, kidney cancer, liver cancer, lung cancer, nasopharangeal cancer, pancreatic cancer, prostate cancer, skin cancer, stomach cancer, gastric cancer, head and neck cancer, testicular cancer, melanoma, acute myelogenous leukemia, chronic myelogenous leukemia, chronic lymphocytic leukemia, T cell lymphocytic leukemia, and B cell lymphomas, and uterine cancer,
wherein the infection is selected from a bacterial infection, a fungal infection, or a viral infection.

28-30. (canceled)

31. The method of claim 12, wherein the subject is suffering from or at risk of suffering from conditions and symptoms caused by a SARS-CoV-2 related viral infection,
wherein administration of the composition results in one or more of:
reduced levels of ACE2 and SARS-COV-2 virus cell entry,
inhibition of SARS-COV2 S protein induced NF-kB activation,
reduced proinflammatory cytokine release by immune effector cells, and
inhibited endothelial activation and dysfunction.

32-40. (canceled)

41. The method of claim 12, wherein the subject is a human subject.

42-45. (canceled)

46. An analgesic pharmaceutical composition for use in treating pain in a subject, wherein the analgesic pharmaceutical composition comprises the composition of claim 1.

47. The analgesic pharmaceutical composition of claim 46, wherein the analgesic pharmaceutical composition is configured for intramuscular injection, subcutaneous injection, intravenous injection, intrathecal injection, sublingual ingestion, skin patch, implantable osmotic pump, collagen implant, aerosol inhalation, and/or suppository.

48. The composition of claim 46, wherein a therapeutically effective amount of the analgesic pharmaceutical composition is used for treating and/or preventing pain in a subject.

49. (canceled)


SEQUENCE LISTING (27 sequences)

SEQ ID NO: 1 - protein, 6 aa, Artificial sequence
  synthetic
EEVVAC

SEQ ID NO: 2 - protein, 4 aa, Artificial sequence
  synthetic
SDKP

SEQ ID NO: 3 - protein, 13 aa, Artificial sequence
  synthetic
CGYGPKKKRKVGG

SEQ ID NO: 4 - protein, 5 aa, Artificial sequence
  synthetic
DDDDD

SEQ ID NO: 5 - protein, 6 aa, Artificial sequence
  synthetic
DDDDDD

SEQ ID NO: 6 - protein, 8 aa, Artificial sequence
  synthetic
NPNANPNA

SEQ ID NO: 7 - protein, 8 aa, Artificial sequence
  synthetic
VAITVLVK

SEQ ID NO: 8 - protein, 6 aa, Artificial sequence
  synthetic
VGVRVR

SEQ ID NO: 9 - protein, 4 aa, Artificial sequence
  synthetic
VIHS

SEQ ID NO: 10 - protein, 5 aa, Artificial sequence
  synthetic
VPDPR

SEQ ID NO: 11 - protein, 4 aa, Artificial sequence
  synthetic
VTCG

SEQ ID NO: 12 - protein, 20 aa, Artificial sequence
  synthetic
SCVRACGADSYEMEEDGVRK

SEQ ID NO: 13 - protein, 19 aa, Artificial sequence
  synthetic
VWSYGVTVWELMTFGSKPY

SEQ ID NO: 14 - protein, 9 aa, Artificial sequence
  synthetic
RMFPNAPYL

SEQ ID NO: 15 - protein, 19 aa, Artificial sequence
  synthetic
SGQARMFPNAPYLPSCLES

SEQ ID NO: 16 - protein, 23 aa, Artificial sequence
  synthetic
SGQARMFPNAPYLPSCLESQPTI

SEQ ID NO: 17 - protein, 6 aa, Artificial sequence
  synthetic
RPAPGS

SEQ ID NO: 18 - protein, 7 aa, Artificial sequence
  synthetic
PPAHGVT

SEQ ID NO: 19 - protein, 5 aa, Artificial sequence
  synthetic
PDTRP

SEQ ID NO: 20 - protein, 19 aa, Artificial sequence
  synthetic
TVCGPKKSTNLVKNKCVNF

SEQ ID NO: 21 - protein, 8 aa, Artificial sequence
  synthetic
LSFELLHA

SEQ ID NO: 22 - protein, 10 aa, Artificial sequence
  synthetic
TNGVGYQPYR

SEQ ID NO: 23 - protein, 17 aa, Artificial sequence
  synthetic
CNGVEGFNCYFPLQSYG

SEQ ID NO: 24 - protein, 24 aa, Artificial sequence
  synthetic
NYLYRLFRKSNLKPFERDISTEIY

SEQ ID NO: 25 - protein, 21 aa, Artificial sequence
  synthetic
SNNLDSKVGGNYNYLYRLFRK

SEQ ID NO: 26 - protein, 36 aa, Artificial sequence
  synthetic
VYADSFVIRGDEVRQIAPGQTGKIADYNYKLPDDFT

SEQ ID NO: 27 - protein, 24 aa, Artificial sequence
  synthetic
SASFSTFKCYGVSPTKLNDLCFTN